SEQUENCE LISTING (15 sequences)

SEQ ID NO: 1 - DNA, 25 bp, Artificial sequences
  Description of Artificial Sequence Synthetic primer
gtgtgtaaatgtttgtatatgtgtg

SEQ ID NO: 2 - DNA, 25 bp, Artificial Sequence
  Description of Artificial Sequence Synthetic primer
aactttcaaataaatacctaaaccc

SEQ ID NO: 3 - DNA, 378 bp, Homo sapiens
cgctgaacttctgggaactggcatttagacggactccgtctcgccgccccccactccctccatggccccacgagccacctccacagcctcgcgccttcccggggtcgcaacgcctatcacaagcttctacccgggtttcagggggcccctgtagtggcgtgggcgccgcgccggtgccgggagaccccctcgtgcgccctctgcggtcggggcagaccctcagccggagcttcttcctcccccacccctgcaggccgggcccaagctgcgacacccccagcacgggctcagcctgtgaacaaactccgagctcagtactcggaggaactggcgagagcgggacggctacctgcccgcacgcccgatttccgggcacag

SEQ ID NO: 4 - DNA, 25 bp, Artificial Sequence
  Description of Artificial Sequence Synthetic primer
tcgtttcgttttcgttagttttttc

SEQ ID NO: 5 - DNA, 22 bp, Artificial Sequence
  Description of Artificial Sequence Synthetic primer
gataccgaaaaaccccctcgta

SEQ ID NO: 6 - DNA, 26 bp, Artificial Sequence
  Description of Artificial Sequence Synthetic primer
tgttttgtttttgttagtttttttga

SEQ ID NO: 7 - DNA, 24 bp, Artificial Sequence
  Description of Artificial Sequence Synthetic primer
ccaataccaaaaaaccccctcata

SEQ ID NO: 8 - RNA, 23 nt, Artificial Sequence
  Description of Artificial Sequence Synthetic oligonucleotide
ucacauaggaaugaaaagccaua

SEQ ID NO: 9 - RNA, 97 nt, Homo sapiens
cacucugcuguggccuauggcuuuucauuccuaugugauugcugucccaaacucauguagggcuaaaagccaugggcuacagugaggggcgagcucc

SEQ ID NO: 10 - DNA, 20 bp, Artificial Sequence
  Description of Artificial Sequence Synthetic primer
gagtggcgggggtaggaggg

SEQ ID NO: 11 - DNA, 20 bp, Artificial Sequence
  Description of Artificial Sequence Synthetic primer
gcaacttcgcgcctaagcgc

SEQ ID NO: 12 - DNA, 192 bp, Artificial Sequence
  Description of Artificial Sequence Synthetic oligonucleotide
gtcgactcacataggaacttaagccataatgctcacataggaacttaagccataatgctcacataggaacttaagccataatgctcacataggaacttaagccataatgctcacataggaacttaagccataatgctcacataggaacttaagccataatgctcacataggaacttaagccataatgtcgac

SEQ ID NO: 13 - DNA, 1790 bp, Homo sapiens
ctggacacattggaatgccttggaaatagaaagaagccatatatgaccagaagccttggaaccagccccatcagaacctgagctattttcctctggccgcagaggtgtaggggtggaatgagccgcggggaagctggctttgaaacctcagggctgtcccagccccggcaagccacaggaaggaggggagagacaggcagcccagcagtgtggagaccctgccacagccagaggagggcagagggagaatccaagggttgagagccagtggcgggtgatggccagcccctggggcccagcccctgtttactggttcttgcaaatgggagctgagcagcctctggacagccagtgacctttgacctcggtgaccactcttctttaagccatagaccctgaggccctgggctgggtgctgggaagggagggttgaaaccaccgtgaaccagagggtgtggctttccaggcaccctcagggagcctccccatctgtccagctggggccagaggctgggagtccctacctgcttcacgttggccggcggctactctggaatgtttttccctccccagaatcaagcttttgcttgatccagaagagcccatatcactaagatggcatatatgtgatctgggcattttcctcctctgcctacagccaggtttagcggcaaacctttcccccttagcaccttcagggctgagttctgggtttctagaggtcaggacggctcctcagagcgccaggaagccagagccccaagcaggacgaaaaagaggcatacacacagcagtgtgaatagcctggccaccagccatcctccctccacctcaagacccccatttgtcccagactaaaggatccagagagcagctccctttctcaggagcttgggcagtgccccagggagtccagggtttctctgcagatgtgcggagcgggaggcggtggtagagagagataaaaggtggagtttctctgttgtttggttcagggattttatttttaattttatgagacagggtcttgctctgtcccccaggctggagtgcagtggcatgatcatagctcactgcagcctcatactcctgggctcaagcaatcctcctgcctcagccttccaactagctgggactacaggtgcgcgccaccgtgcctggctaacttttcattttttttgtagggacggggtctcgttttgttgccaaagctggtctcaaacttgtggcctcaagcaatccacctgccttggcctcccaaagtgctgagattgcagatgtgagccaccgtgcctggccagatttttcttttattcttctttctttttcttttttgctttcttgtcttttcagaagcaagccagacctagcaggctgttccatgttctatttttgactgtagccacagctgctgttctcaggacagcatcccttcccacatgcctgcgcctgctgcctgctgagatgaggaggggagcgtctgggaacttgcgagtccaaggccagtccccatttctgcctcgctcaccgctggcccttagagaccccgaggtaggggtggggagatgcttctctccttgccccccgccctcatgggtcctagcccttccctgagtgcgggctgaggccagagtcaccttttctgtggctggctctaccttcctgtccctgaggttaaacggtgcccatcctgccatcctcaaacgacagaggagcttttctggaatttcaaaccattgctcttagtcccaagctaggcttaaacctggaatctacaagccaaaagtccc

SEQ ID NO: 14 - DNA, 1083 bp, Homo sapiens
gacgtctggtgtgtggagagtactgcatgagcagagttcttctattataaaattaccatatcttgccattcacagcaggtcctgtgaatacgtttttactgagtgtctttaaatgaggtgttctagacagtgtgctgataatgtattgtgcgggtgacctcttcgctatgattgtatctcttactgttttgttaaagaaatgcagatgtgtaactgagaagtgatttgtgtgtgtgtcttggttgtgattggattctttggggggggggaactgaaacatttgtcatatactgaacttatatacatcaaaagggattaatacagcgatgccaaaaagtttaatcacggacacatgtccgtttctgtagtccgtatgctctttcattcttggtagagctggtatgtggaatgccatacctctgaccctactacttacctttttactgacagactgcccacactgaaagcttcagtgaatgttcttagtcctgttttcttctgttactgtcaggaaactgagtgatctaatggttctctcactttttttttgttcttttagtgtactttgaagtatcaaatcttaacttggtttaaacaatacatattcctaacctttgtaaaaaagcaaagattcttcaaaatgacattgaaataaaaagtaagccatacgtattttcttagaagtatagatgtatgtgcgtgtatacacacacacacacacacacagagataaacacaatattccttatttcaaattagtatgattcctatttaaagtgatttatatttgagtaaaaagttcaattcttttttgctttttaaaaaatctgatgcttcataattttcattatattattccacatatttttccttgaagttcttagcataatgtatccattacttagtatatatctaggcaacaacacttagaagtttatcagtgtttaaactaaaaaaataaagattcctgtgtactggtttacatttgtgtgagtggcatactcaagtctgctgtgcctgtcgtcgtgactgtcagtattctcgctattttatagtcgtgccatgttgttactcacagcgctc

SEQ ID NO: 15 - DNA, 60 bp, Homo sapiens
gcgtgggcgccgcgccggtgccgggagaccccctcgtgcgccctctgcggtcggggcaga

(19) United States
(12) Patent Application Publication
(10) Pub. No.: US 2015/0141485 A1
(43) Pub. Date: May 21, 2015

(54) PROGNOSIS AND TREATMENT OF LUNG CANCER USING miRNA-135b

(71) Applicants: NATIONAL CHENG KUNG UNIVERSITY, TAINAN CITY (TW); NATIONAL TAIWAN UNIVERSITY, TAIPEI CITY (TW)

(72) Inventors: Pan-Chyr Yang, Taipei City (TW); Ching-Wen Lin, Taipei City (TW); Tse-Ming Hong, Tainan City (TW); Yih-Leong Chang, Taipei City (TW)

(73) Assignees: NATIONAL CHENG KUNG UNIVERSITY, TAINAN CITY (TW); NATIONAL TAIWAN UNIVERSITY, TAIPEI CITY (TW)

(21) Appl. No.: 14/085,323

(22) Filed: Nov. 20, 2013

(51) Int. Cl. C12Q 1/68; C12N 15/113

(52) U.S. Cl. CPC C12Q 1/6886; C12N 15/113
USPC 514/44 A; 435/6.12; 435/6.11

(57) ABSTRACT

The present invention provides a method for the prognosis of lung cancer patient based on the expression levels of miRNA-135b, LZTS1, LATS2 and nuclear TAZ. The invention also provides a method for treatment of lung cancer.

### BACKGROUND OF THE INVENTION

1. Field of the Invention

The invention relates to a method for the prognosis of lung cancer, and in particular, to the method for the prognosis using the expression of miRNA-135b.

2. The Prior Arts

Lung cancer causes more deaths worldwide than any other form of cancer, in many countries, it is the primary cause of cancer death among both men and women. Lung cancer is a disease characterized by uncontrolled cell growth in tissues of the lung. Most cancers that start in lung, known as primary lung cancers, are carcinomas that derive from epithelial cells. The main types of lung cancer are small-cell lung carcinoma (SCLC), also called oat cell cancer, and non-small-cell lung carcinoma (NSCLC). NSCLC is any type of epithelial lung cancer other than small cell lung cancer (SCLC). The most common types of NSCLC are squamous cell carcinoma, large cell carcinoma, and adenocarcinoma, but there are several other types that occur less frequently, and all types can occur in unusual histologic variants.

MicroRNAs (miRNAs) are a class of small, non-coding RNAs that can repress the expression of multiple target genes through the endogenous RNA interference machinery. The miRNAs can regulate a wide range of cellular functions including proliferation, apoptosis, differentiation and development. Some miRNAs exert only minor effects on target gene repression, however, increasing evidence suggests that miRNAs can confer robustness of biological processes via regulation of target networks. For example, miR-126 impacts endothelial recruitment by modulating the IGF1/IGF1R and GAS6/MERK pathways. Recent studies have also shown that dysregulation of miRNAs is involved in carcinogenesis and metastasis in several human cancer types.

Lung cancers can develop a high metastatic potential, which is the major cause of treatment failure. Several miRNAs, including miR-126, miR-21 and miR-335, have been associated with metastasis in several types of cancers.

### SUMMARY OF THE INVENTION

The present invention provides a method of determining the prognosis of a subject with lung cancer, comprising:
a. measuring the expression level of miRNA-135b in a test sample from a subject with lung cancer; and
b. determining the prognosis of the subject with lung cancer, wherein a high expression level of miR-135b in the test sample compared to noncancerous lung tissue control indicates an adverse prognosis.

In one aspect, the present invention provides the method of determining the prognosis of a subject with lung cancer, further comprises a step of measuring the expression level of LZTS1, wherein the expression level of LZTS1 in the test sample compared to noncancerous lung tissue control less than 0.25 fold indicates an adverse prognosis.

In another aspect, the present invention provides the method of determining the prognosis of a subject with lung cancer, further comprises a step of measuring the expression levels of LZTS1 and LATS2, wherein decreased expression levels of LZTS1 and LATS2 in the test sample compared to noncancerous lung tissue control indicate an adverse prognosis.

In another aspect, the present invention provides the method of determining the prognosis of a subject with lung cancer, further comprises a step of measuring at least one additional gene selected from the group consisting of LZTS1, LATS2 and nuclear TAZ expression, wherein decreased expression levels of LZTS1 and LATS2 compared to noncancerous lung tissue control indicate an adverse prognosis, and increased expression level of nuclear TAZ in the test sample compared to noncancerous lung tissue control indicates an adverse prognosis.

In another aspect, the present invention provides the method of determining the prognosis of a subject with lung cancer, wherein the lung cancer is non-small-cell lung cancer (NSCLC).

In another aspect, the present invention provides the method of determining the prognosis of a subject with lung cancer, wherein a prediction of prognosis is given by a likelihood score derived from using Kaplan-Meier survival analysis, wherein the performance of miRNA-135b of subject is assessed.

In another aspect, the present invention provides the method of determining the prognosis of a subject with lung cancer, further comprising a step of performing a Kaplan-Meier survival analysis, wherein the performance of at lest one miRNA-135b and LZTS1 of subject is assessed.

In another aspect, the present invention provides the method of determining the prognosis of a subject with lung cancer, further comprising a step of performing a Kaplan-Meier survival analysis, wherein the performance of at least one of miRNA-135b, LZTS1 and LATS2 of subject is assessed.

In another aspect, the present invention provides the method of determining the prognosis of a subject with lung cancer, further comprising a step of performing a Kaplan-Meier survival analysis, wherein the performance of at least one of miRNA-135b, LZTS1, LATS2 and nuclear TAZ of subject is assessed.

In another aspect, the present invention provides the method of determining the prognosis of a subject with lung cancer, wherein the adverse prognosis indicates growth, invasion, migration and metastasis of lung cancer.

The present invention also provides a method of inhibiting growth, invasion, migration and metastasis of lung cancer in a subject, which comprises administering the subject with an effective amount of miRNA sponge or miRNA antagomir.

In one aspect, the present invention provides the method of inhibiting growth, invasion, migration and metastasis of lung cancer in a subject, wherein the miRNA sponge is miR-135b-specific molecular sponge the miRNA sponge is miR-135b-specific molecular sponge, the miRNA antagomir is miR-135b-antagomir, and the lung cancer is non-small-cell lung cancer (NSCLC).

The present invention also provides a method of assaying and/or identifying a test agent as a regulator of a methylation level of miRNA-135b for treatment lung cancer, comprising:
a. providing a cell comprising a CpG island of the miRNA-135b promoter region, and treating the cell with the test agent or a vehicle control;
b. measuring the methylation level in the CpG island of the miRNA-135b promoter region, and calculating the ratio of the methylation level of the miRNA-135b promoter region in the presence and the absence of the test agent; and
c. identifying the test agent as a regulator of the methylation level of miRNA-135b when the ratio in the presence of the test agent is more than that in the vehicle control.

In one aspect, the present invention provides determining the prognosis of a subject with lung cancer, wherein the lung cancer is non-small-cell lung cancer (NSCLC).

In another aspect, the present invention provides determining the prognosis of a subject with lung cancer, wherein the CpG island of miR-135b promoter region contains NF-κB (nuclear factor kappaB) binding site.

In another aspect, the present invention provides determining the prognosis of a subject with lung cancer, wherein the step b comprises differential methylation hybridization (DMH) microarray screening, methylation-specific polymerase chain reaction (MSP), quantitative methylation-specific polymerase chain reaction (QMSP), bisulfite sequencing (BS), micro arrays, mass spectrometry, denaturing high-performance liquid chromatography (DHPLC), and pyrosequencing.

In another aspect, the present invention provides determining the prognosis of a subject with lung cancer, wherein lower degree of methylation of CpG island of miRNA-135b promoter region indicates an adverse prognosis and the adverse prognosis indicated growth, invasion, migration and metastasis of lung cancer.

### BRIEF DESCRIPTION OF THE DRAWINGS

FIG. 1 is a schematic model of lung cancer invasion and metastasis by miR-135b regulatory axis. The expression level of miR-135b is regulated by DNA methylation and NF-κB (nuclear factor kappaB) activity. Once miRNA-135b is upregulated, it suppresses downstream target genes such as LZTS 1 and Hippo pathways, therefore promotes tumor growth, EMT (epithelial-mesenchymal-transition), invasion/metastasis and caner stemness.

FIG. 2a shows miR-135b up-regulated in a highly invasive lung cancer cell line and modulated cell invasion and migration ability. Real-time RT-PCR was conducted to quantify the endogenous expression of miR-135b, miR-21 and miR-126* in CL-series cell lines. Assays were performed in triplicate, and the results are presented as the fold-change in expression compared with CL1-0. The expression of U6B was used as a normalization control.

FIG. 2b shows miR-135b upregulated in a highly invasive lung cancer cell line and modulated cell invasion and migration ability. Migration assays were performed in CL1-0 cells transduced with control (Neo) or miR-135b-expressing (miR-135 M5) lentiviral vectors. The number of migrating cells in three different fields was counted 15 and 24 hours after the inserts were removed. *P<0.05;**P<0.005 by Student's t-test.

FIG. 2c shows miR-135b upregulated in a highly invasive lung cancer cell line and modulated cell invasion and migration ability. An invasion assay was performed in lentiviral vector-modified CL1-0 cells as described in FIG. 2b. The number of invading cells was counted 20 hours after cell seeding and is presented as the mean±s.d.

FIG. 2d and FIG. 2e show miR-135b upregulated in a highly invasive lung cancer cell line and modulated cell invasion and migration ability. CL1-5-F4 and Hop-62 cells were transfected with scramble (NC) of miR-135b-specific antagomir (Antago-135b) for 48 hours and the subjected to migration assays (FIG. 2d) and invasion assays (FIG. 2e) as described above. *P<0.05;**P<0.005 by Student's t-test.

FIG. 2f shows miR-135b upregulated in a highly invasive lung cancer cell line and modulated cell invasion and migration ability. CL1-0 cells transduced with control (Neo) or miR-135b-expressing (miR-135b) lentiviral vectors were subjected to a soft agar assay.**P<0.005 by Student's t-test.

FIG. 2g shows miR-135b upregulated in a highly invasive lung cancer cell line and modulated cell invasion and migration ability. CL1-5-F4 and Hop-62 cells were transfected with the negative control (NC) or the Anatago-135b for 48 hours and subjected to soft agar assays. Error bars indicate mean±s.d.,*P<0.05 by Student's t-test.

FIG. 3a shows that miR-135b promotes xenograft tumor growth and metastasis in a mouse model. Nude mice were subcutaneously injected with 1.2×10⁶ CL1-0 cells that stably expressed either control-vector (Neo) or miR-135b lentiviral vector. After implantation of 7 days, tumor volume measurement began and was performed every 4 days. (n=9); Mean±s.e.m.,*P<0.05;**P<0.005 by Student's t-test.

FIG. 3b shows that miR-135b promotes xenograft tumor growth and metastasis in a mouse model. CL1-0 cells that were transduced with control (Neo) or miR-135b-expressing lentriviral vectors were intravenously injected into NOD-SCID mice (10 mice per group). The statistical incidence of lung or soft tissue tumor generation in the mice after injection with CL1-0 cells with different vector-transduced cells. The animals were killed 8 weeks each group after injection, and lung sections were examined by hematoxylin/eosin (H&E) staining. Scale bar: 200 μm.

FIG. 3c shows that miR-135b promotes xenograft tumor growth and metastasis in a mouse model. The number of lung tumor nests in each group was counted under a low power field (LPF) and is presented as the mean±s.d., **P<0.005 by Student's t-test.

FIG. 3d shows that miR-135b promotes xenograft tumor growth and metastasis in a mouse model. CL1-5-F4 cells transfected with plasmids expressing either control or miR-135b sponges were analyzed using in vitro invasion assay. The average number of invading cells obtained in the transwell invasion assay is presented (n=3).

FIG. 3e shows that miR-135b promotes xenograft tumor growth and metastasis in a mouse model. CL-5-F4 cells stably expressing control sponge or miR-135b sponge were intravenously injected into SCID mice (n=8). Representative pictures of murine whole lung (left) and H&E staining of lung sections (right) are presented.

FIG. 3f shows that miR-135b promotes xenograft tumor growth and metastasis in a mouse model. Lung tumor nests in each group were examined and counted using a microscope (n=8); Mean±s.e.m. is shown,*P<0.05 by Student's t-test.

FIG. 4a shows that systemic delivery of antagomiR-135b inhibits metastasis and tumor growth in vivo. Nude mice were subcutaneously injected with 5×10⁵ H441 cells. Scramble antagomir (NC) or miR-135b Antagomir (antago-135b) was administered via tail vein injection 2 days after implantation. On day 14 after cancer cell implantation, tumor volume measurements began and were performed every 4 days (n=9). Mean±s.e.m. is shown,*P<0.05 by Student's t-test.

FIG. 4b to FIG. 4d show that systemic delivery of antagomiR-135b inhibits metastasis and tumor growth in vivo. CL1-5 cells were implanted orthotopically into the left lungs of 6-weeks-old nude mice (n=9). A series of scramble antagomir (NC) or miR-135b antagomir (Antago-135b) intravenous injections were performed every 2 days after tumor cell implantation. Lungs were harvested 25 days after the cells were implanted. FIG. 4b shows bright-field imaging and H&E staining of CL1-5 tumor-bearing lungs. Arrows indicate the visible nodules. FIG. 4c shows primary tumor volume and FIG. 4d shows the number of intra-lung metastatic nodules in CL1-5 tumor-bearing mice treated with scramble antagomir (NC) of miR-135b antagomir (Antago-135b) 25 days after orthotopic implantation. Data are expressed as mean±s.e.m. *P<0.05 by Student's t-test.

FIG. 4e shows that systemic delivery of antagomiR-135b inhibits metastasis and tumor growth in vivo. Mice bearing CL1-5 cells were subjected to scramble antagomir (NC) or miR-135b antagomir (Antago-135b) treatment. Animal survival was determine using Kaplan-Meier survival analysis and the log-rank test for data obtained 25 days after the cancer cells were orthotopically injected (n=9).

FIG. 4f shows that systemic delivery of antagomiR-135b inhibits metastasis and tumor growth in vivo. CL1-5 cells with scramble antagomir (NC) or miR-135 antagomir (Antago-135b) were intravenously injected to assess the effect of antagomir on late-stage metastasis (n=9). Representative average of visible metastatic lung nodules and H&E staining (right). Data are expressed as the mean±s.e.m. *P<0.05 by Student's t-test.

FIG. 5a shows that LZTS1 is a direct target of miR-135b. Co-transduction of CL1-0 cells with a control vector (Ctrl vector) or a miR-135b-expressing plasmid with firefly luciferase fused with 3′UTR sequences of putative miR-135b target genes. Luciferase activity was measured, and the relative ratio of the activity in the miR-135b groups to that in the control vector group is presented as the mean±s.d., *P<0.05 by Student's t-test.

FIG. 5b shows that LZTS1 is a direct target of miR-135b. CL1-0 cells were co-transfected with Ctrl vector- or miR-135b-expressing plasmids and firefly luciferase fused with wild-type (wt) LZTS 1 3′-UTR or seed sequence-mutated (mut) 3′-UTR (1 mut, seed 1 mutated; 2 mut, both seeds mutated). Mean±s.d. is shown, *P<0.05 by Student's t-test.

FIG. 5c shows that LZTS1 is a direct target of miR-135b. CL1-0 cells were co-transfected with miR-135b, wild-type LZTS1 3′-UTR along with Scramble or miR-135-specific antagomir (Antago-135b). Luciferase activity was measured and is presented as described in FIG. 5a. Mean±s.d. is shown, *P<0.05; **P<0.005 by Student's t-test.

FIG. 5d shows that LZTS1 is a direct target of miR-135b. Western blot analysis of endogenous LZTS1 expression in CL1-0 cells after transduction with different multiplicities of infections of control (Neo) or miR-135b-expressing lentiviral vectors.

FIG. 5e shows that LZTS1 is a direct target of miR-135b. CL1-5-F4 and UACC-257 cells were administrated with 100 nM of negative control (NC) or antago-135b for 48 hours. Total cell lysates were harvested for western blot analysis.

FIG. 5f shows that LZTS1 is a direct target of miR-135b. The cells were transduced with different amounts of LZTS1-expressing lentiviral vector, and western blot analysis of LZTS1 expression along with analyses of invasion and migration ability were conducted as described above. Mean±s.d. is shown, *P<0.05; **P<0.005 by Student's t-test.

FIG. 5g shows that LZTS1 is a direct target of miR-135b. UACC-257 cells were transfected with control (NC) or LZTS1-specific siRNAs (si-LATS1 no. 2 and no. 3) for 48 hours and subjected to invasion and migration assays as described above. Mean±s.d. is shown, *P<0.05; **P<0.005 by Student's t-test.

FIG. 5h shows that LZTS1 is a direct target of miR-135b. CL1-0 cells stably expressing control vector, miR-135b, or co-expressing miR-135b and LZTS1 were analyzed in transwell invasion assays. Mean±s.d. is shown, *P<0.05 by Student's t-test.

FIG. 5i shows that LZTS1 is a direct target of miR-135b. The statistical incidence of lung nodule generation in the mice after injecting CL1-0 cells with different vector-transduced cells. The animals were killed 8 weeks after injection (n=6). Mean±s.e.m. is shown, *P<0.05 by Student's t-test.

FIG. 5j shows that LZTS1 is a direct target of miR-135b. The number of colonies of soft agar assay by CL1-0 cell derivatives with Neo-control, miR-135b or miR-135b+LZTS1(n=3). *P<0.05 by Student's t-test.

FIG. 6a shows multiple components of a Hippo pathway regulated by miR-135b. CL1-0 cells were co-transfected with control vector (Ctrl vector) or miR-135b-expressing plasmids with firefly luciferase fused with 3′-UTR sequences of putative miR-135b target gene. Luciferase activity was measured, and the relative ratio of activity in the miR-135b groups to that in the control vector groups is presented as the mean±s.d., *P<0.05; **P<0.01 by Student's t-test.

FIG. 6b shows multiple components of a Hippo pathway regulated by miR-135b. CL1-0 cells co-transfected with miR-135b and pGL3-3′-UTRs with scramble (NC) or Antago-135b for 60 hours. Luciferase activity was assayed and is presented a described in FIG. 6a. Mean±s.d. is shown, *P<0.05; **P<0.005 by Student's t-test.

FIG. 6c shows multiple components of a Hippo pathway regulated by miR-135b. Western bolt analysis of the Hippo pathway components in CL1-0 and HEk-293 cells transduced with control (Neo) or miR-135b lentiviral vectors.

FIG. 6d shows multiple components of a Hippo pathway regulated by miR-135b. Western bolt analysis of the Hippo pathway proteins in CL1-5-F4 and CL141 cells incubated with Scramble (NC) of Antago-135b for 72 hours. Quantitative RT-PCR was used to assay miR-135b knockdown activity.

FIG. 6e shows multiple components of a Hippo pathway regulated by miR-135b. Endogenous TAZ proteins in CL1-0 cells transfected with control (pClneo), miR-135b, miR-135+LATS2. The cells were harvested after 36 hours of transfection.

FIG. 7a is Kaplan-Meier survival analysis of NSCLC (non-small-cell lung carcinoma) patients with different levels of miR-135b expression and its targets. Kaplan-Meier plots of overall survival in 112 NSCLC patients in high- and low-risk groups based on miR-135b expression levels.

FIG. 7b is Kaplan-Meier survival analysis of NSCLC patients with different levels of LZTS1 expression. Expression of LZTS1 was examined via immunohistochemical staining in serial dissections of primary tumor specimens from 147 NSCLC patients who underwent surgical resections. Patients were designated as having high LZTS1 expression if more than 50% of the neoplastic cells in the tumor sections had positive immunoreactivity, and as having low LZTS1 expression if fewer than 50% of the cells were immunoreactive. The result reflected Kaplan-Meier estimates of overall survival for NSCLC patients according to the expression of LZTS1. P values were obtained from two-sided log-rank tests.

FIG. 7c is Kaplan-Meier survival analysis of NSCLC patients with different levels of LATS2 expression. Expression of LATS2 was examined via immunohistochemical staining in serial dissections of primary tumor specimens from 147 NSCLC patients who underwent surgical resections. Patients were designated as having high LATS2 expression if more than 50% of the neoplastic cells in the tumor sections had positive immunoreactivity, and as having low LATS2 expression if fewer than 50% of the cells were immunoreactive. The result reflected Kaplan-Meier estimates of overall survival for NSCLC patients according to the expression of LATS2. P values were obtained from two-sided log-rank tests.

FIG. 7d is Kaplan-Meier survival analysis of NSCLC patients with different levels of TAZ expression. Expression of TAZ was examined via immunohistochemical staining in serial dissections of primary tumor specimens from 147 NSCLC patients who underwent surgical resections. The patients were designated as having high nuclear TAZ expression if more than 50% of the neoplastic cells had a positive TAZ signal in the nucleus, and as having low nuclear TAZ expression if fewer than 50% of the neoplastic cells had a positive TAZ signal in the nucleus. The result reflected Kaplan-Meier estimates of overall survival for NSCLC patients according to the expression of nuclear TAZ. P values were obtained from two-sided log-rank tests.

FIG. 7e is Kaplan-Meier survival analysis of NSCLC patients with different levels of LZTS1 and LATS2 co-expressions. Co-expressions of LZTS1 and LATS2 were examined via immunohistochemical staining in serial dissections of primary tumor specimens from 147 NSCLC patients who underwent surgical resections. The result reflected Kaplan-Meier estimates of overall survival for NSCLC patients according to the expressions of both LZTS1 and LATS2. P values were obtained from two-sided log-rank tests.

FIG. 7f shows in situ hybridization of miR-135b (upper panel) and immunohistochemical analysis of LZTS1 (lower panels) expression in serial sections of NSCLC tumor specimens. Scale bar: 100 μm.

FIG. 8a shows dual regulations of the expression miR-135b by DNA demethylation and NF-κB signaling. Bisulfite sequencing analysis was performed in CL-series cells. Each square represents a CpG dinucleotide, and the colors indicate the percentage of methylation.

FIG. 8b shows dual regulations of the expression miR-135b by DNA demethylation and NF-κB signaling. Quantitative methyaltion-specific PCR (qMS-PCR) was performed to analyze the amount of methylated (M) and unmethylated (U) DNA in CL-series cells. The ratio of M to U in each cell line was calculated and is presented as the mean±s.d. of the ratios compared with M to U ratio in CL1-0 cells.

FIG. 8c shows dual regulations of the expression miR-135b by DNA demethylation and NF-κB signaling. Cells were treated with different doses of 5-aza-2′-CdR (5′-aza) for 4 days, and total RNA was harvested for real time RT-PCR. Data are expressed as the mean±s.d.

FIG. 8d shows dual regulations of the expression miR-135b by DNA demethylation and NF-κB signaling. The cells were treated with indicated amounts of 5-aza-2′-CdR (upper panel) for 4 days or TNF-α (lower panel) for 6 hours before being subjected to chromatin immunoprecipitation (ChIP) with anti-p65 antibody and specific primers.

FIG. 8e shows dual regulations of the expression miR-135b by DNA demethylation and NF-κB signaling. CL1-5-F4 cells were treated with different concentrations of TNF-α with or without BAY-117082 (5 or 10 μM) for 6 hours. Total RNA was harvested, and miR-135b expression was analyzed and analysis by real-time RT-PCR. Mean±s.d. is shown, *P<0.05; **P<0.005 by Student's t-test.

FIG. 8f shows dual regulations of the expression miR-135b by DNA demethylation and NF-κB signaling. Cells were treated with or without 5-aza-2′-CdR for 4 days and re-seeded for TNF-α stimulation for 6 hours. Total RNA was harvested, and miR-135b expression was analyzed. Mean±s.d. is shown, *P<0.05; **P<0.005 by Student's t-test.

FIG. 9 shows schematics of the 10 GpC sites in the NF-κB binding region. The shaded region indicates the NF-κB binding site in the promoter region of miR-135b.

### DETAILED DESCRIPTION OF THE INVENTION

The terms used in this specification generally have their ordinary meanings in the art, within the context of the invention, and in the specific context where each term is used. Certain terms that are used to describe the invention are discussed below, or elsewhere in the specification, to provide additional guidance to the practitioner regarding the description of the invention.

Unless otherwise defined, all technical and scientific terms used herein have the same meaning as commonly understood by one of ordinary skill in the art to which this invention pertains. In the case of conflict, the present document, including definitions will control.

As used herein interchangeably, a “miR gene product,” “microRNA,” “miR,” or “miRNA” refers to the unprocessed or processed RNA transcript from a miR gene.

As used herein, “approximately” shall generally mean within 20 percent, preferably within 10 percent, and more preferably within 5 percent of a given value or range. Numerical quantities given herein are approximate, meaning that the term “approximately” can be inferred if not expressly stated.

The level of the at least one miR gene product can be measured using a variety of techniques that are well known to those of skill in the art (i.e., quantitative or semi-quantitative RT-PCR, Northern blot analysis, solution hybridization detection).

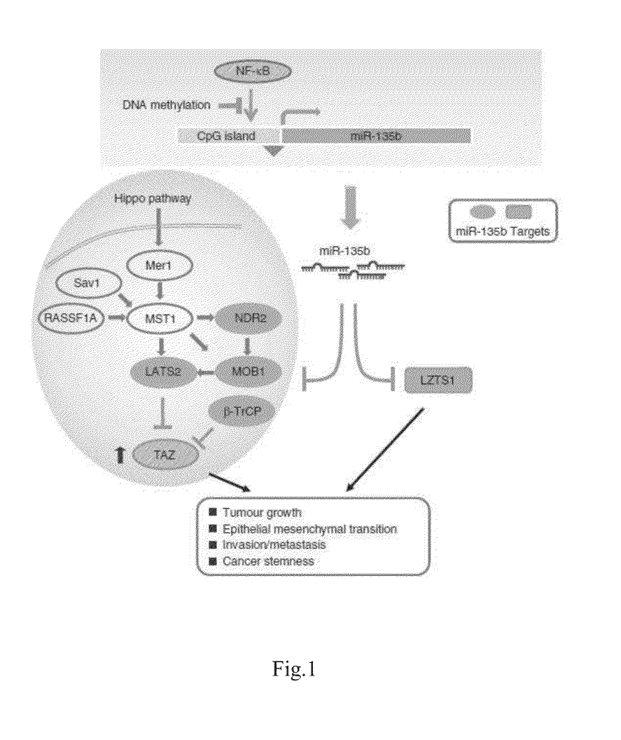

In present invention, as shown in FIG. 1, miRNA-135b is overexpressed in lung cancer cells, and miR-135b acts as an oncogenic miRNA that promotes tumor growth and cellular invasiveness and metastasis in lung cancer, enforcing its oncogenic function through the repression of multiple key components of a Hippo pathway network along with the tumor suppressor LZTS1. And a miRNA-135b antagomir (Antago-135), which is able to functionally suppress miR-135b, effectively reduces metastasis and tumor burden, which suggests the potential for the development of mir-135b antagonists for lung cancer therapy.

In one embodiment, the function of LZTS1 is to suppress the migratory and invasive activity of tumor cells in lung cancer. LZTS1 expression in tumor specimens is predictive of overall survival of NSCLC patients. And LZTS1 also overexpressed in CL1-5 cells, the epithelial-mesenchymal-transition (EMT) regulator of Slug protein expression is suppressed, while knockdown of LZTS1 in CL1-0 cells upregulates the Slug protein expression. Consistent with the finding that miR-135b can modulate the EMT marker; miRNA-135b may control cancer invasion and metastasis through downregulating LZTS1 in lung cancer cells.

In one embodiment, a series of miR-135b target genes is functioned as tumor suppressors and belongs to a Hippo signaling pathway. The mammalian Hippo pathway is mainly composed of a kinase cascade that includes MST1/2, LATS1/2, MOB1a/b, and Sav1, which phosphorylates the transcriptional coactivator TAZ/YAP. Phospho-TAZ protein retained in the cytoplasm is recognized by SCT-^{β-TRCP}-mediated degradation. NDR1/2 shares a similar NDR domain with LATS1/2 and is phosphorylated by MST1/2 and MOB1, indicating that it may have an extended role in Hippo tumor suppressor pathways. The Hippo pathway prevents overgrowth of organs and it has also been shown to suppress tumor growth by inhibiting TAZ. The present invention disclosed that miR-135b affected Hippo-related pathways by downregulating the levels of LATS2, NDR2, MOB1b, and β-TrCP proteins. The variations in the downregulation levels of Hippo components in different cell lines indicate that these proteins are not coincidentally regulated by miR-135b. However, the expression levels of TAZ in lung cancer cell lines are consistently with miR-135b. In some cases, TAZ/YAP has been shown to not be influenced by LATS2 in specific cell types, however, the N-terminal phosphodegron of TAZ has been shown to be phosphorylated by GSK-3β and mediated by β-TrCP in a LATS2-independent manner. It is worth noting that TAZ contributes to anchorage-independent growth and EMT in immortalized mammalian cells by driving the activation of a set of genes. Thus, miR-135b may be able to affect functions similar to those regulated by TAZ. Namely, miR-135b may participate in the Hippo pathway, potentially presiding over limitless growth of tumors by stabilizing the TAZ/YAP protein via the regulation of a variety of different targets.

In one embodiment, CpG islands on the miR-135b promoter region are highly methylated in low-invasive cancer cells, and that a DNA demethylating agent can increase miR-135b expression. A TNF-α-stimulated NF-κB (nuclear factor kappaB) signaling cascade synergistically acts with DNA demethylation to further elevate miR-135b expression. Quantitative pyrosequencing analysis reveals that degree of methylation of the putative of NF-κB binding sites on the miR-135b promoter is inversely related to the levels of miR-135b expression. DNA methylation may prevent NF-κB from bind to the miR-135b promoter. Therefore, microenvironment stimulates, such as inflammatory cytokines, are exploited by cancer cells so that endogenous epigenetic mechanism acquire metastatic ability through modulation of miRNA expression.

### EXAMPLES

The method of determining the risk of developing a tumor requires that a sample be taken from a human. The sample comprises tissue sample, which includes, but not limited to, epithelial tissue, connective tissue, muscle tissue and nervous tissue. The epithial tissue samples include simple epithelia (i.e., squamous, cuboidal and columner epithelium), pseudo-stratified epithelia (i.e., columnar) and stratified epithelia (i.e., squamous). The connective tissue samples include embryonic connective tissue (i.e., mesenchyme and mucoid), ordinary connective tissue (i.e., loose and dense), and special connective tissue (i.e., cartilage, bone, and adipose). The muscle tissue sample include smooth (i.e., involuntary) and striated (i.e., voluntary and involuntary). The nervous tissue sample includes neurons and supportive cells. In addition, the sample may contain cells unique to the pulmonary system, such as cells from the trachea, bronchi, bronchioli, and alveoli. Cells unique to the mouth and throat are also included such as all cell types exposed in the mouth that include cheek lining, tongue, floor and roof of the mouths, gums, throat as well as sputum samples.

The method also requires that a normal sample be taken from a human. The normal sample comprises tissue samples, such as epithelial tissue, connective tissue, muscle tissue and nervous tissue. The epithial tissue samples include simple epithelia (i.e., squamous, cuboidal and columner epithelium), pseudo-stratified epithelia (i.e., columnar) and stratified epithelia (i.e., squamous). The connective tissue samples include embryonic connective tissue (i.e., mesenchyme and mucoid), ordinary connective tissue (i.e., loose and dense), and special connective tissue (i.e., cartilage, bone, and adipose). The muscle tissue sample includes smooth (i.e., involuntary) and striated (i.e., voluntary and involuntary). The nervous tissue sample includes neurons and supportive cells. In addition, the sample may contain cells unique to the pulmonary system, such as cells from the trachea, bronchi, bronchioli, and aveoli. Cells unique to the mouth and throat are also included such as all cell types exposed in the mouth that include cheek lining, tongue, floor and roof of the mouths, gums, throat as well as sputum samples.

### 1. Materials and Methods

### (a) Cell Culture and Antibodies

The lung cancer cell lines CL1-0, CL1-1, CL1-5 and CL1-5-F4 were derived from in vitro transwell and in vivo metastasis selection as previously described (Chu, Y. W., et al. 1997). A549, HOP-62, H441, and CL141 cells and melanoma cell line UACC-257 were maintained in RPMI medium supplemented with 10% fetal bovine serum (FBS). The H1299 and HEK-293 cells were maintained in DMEM (Dulbecco's Modified Eagle Medium) with 10% FBS.

The primary antibodies used for immunoblot analysis and immunohistochemical staining were mouse anti-LZTS1 (Abnova, Taipei, Taiwan), mouse anti-HA antibody (Covance Inc., CA USA), rabbit anti-LATS2 (Bethyl Laboratories, INC., Cambridge, UK), rabbit anti-βTrCP (Cell Signaling Technology, INC., MA, USA), rabbit anti-NDR2 (Santa Cruz Biotechnology, CA, USA), rabbit anti-TAZ (Cell Signaling Technology), and mouse anti-β-actin antibody (Santa Cruz Biotechnology).

Cells were seeded at concentration of 1×10⁵ for treatment with 5-aza-2′-CdR (R&D Systems INC., MN, USA) for 96 hours. For TNF-α (PeproTech, Rocky Hill. N.J.) stimulation, 1×10⁶ cells were seeded for 24 hours and treated with different amounts of TNF-α for 6 hours.

### (b) Lentiviral Vector Transduction

Pre-miR-135b-encoding sequences and LZTS1-encoding sequences were subcloned into the pLKO-AS2.neo vector (obtained from the National RNAi Core Facility in Academia Sinica, Taipei, Taiwan), and lentiviral vectors were prepared in accordance with standard protocols. CL1-0, UACC-257, and A549 cells were infected by lentiviruses with different multiplicities of infection in medium containing polybrene. One day after infection, the cells were treated with G418 to drive a pool of neomycin-resistant clones.

### (c) Bisulfite Sequencing and qMS-PCR (Quantitative MS-PCR)

For the bisulfite sequencing, the genomic DNA was treated with sodium bisulfite as describe in the manual (Zymo Research, Orange, Calif.). The bisulfite-treated DNA was desalted and eluted in an elution buffer. Next, DNA was amplified with the forward primer mir-135-BF (SEQ ID NO:1) and the reverse primer mir-135-BR (SEQ ID NO:2). The PCR products were ligated into the TA cloning vector (RBC Bioscience, Taipei, Taiwan) and analyzed to determine the DNA sequence.

For qMS-PCR, the genomic DNA was converted with an EZ DNA methylation kit (Zymo Research). Modified DNA was then subjected to real-time quantitative methylation PCR as previous described (Chan, M. W., et al. 2005). The primers target the miR-135b promoter (SEQ ID NO:3) were as follow: the forward primer of methylated promoter 135b-M2F (SEQ ID NO:4), the reverse primer of methylated promoter 135b-M2R (SEQ ID NO:5), the forward primer of unmethylated promoter 135b-U2F (SEQ ID NO:6) and the reverse primer of unmethylated promoter 135b-U2R (SEQ ID NO:7).

### (d) Quantitative PCR Analysis

Total RNA was isolated using TRIZOL reagent (Invitrogen, Carisbad, Calif.) according to the standard protocol. The mature miR-135b and endogenous control U6B were analyzed using TaqMan Micro RNA Assay (Applied Biosystems, Foster City, Calif.). Briefly, total RNA was reverse-transcribed via SuperScipt-III Reverse Transcriptase (Invitrogen, Carlsbad, Calif.). The cDNA was amplied with TaqMan 2× Universal Master Mix (Applied Biosystems), and miRNA-specific real-time PCR was performed using an ABI 7500 real-time PCR system.

### (e) Luciferase reporter assay

One day before transfection, CL1-0 cells were seeded at a concentration of 2.5×10⁴ per well. Next, the pClneo vector or miR-135b plasmid was co-transfected with the pGL3-target gene-3′UTR. The Renilla lunciferase plasmid (phRL-TK, Promega, Madison, Wis.) was co-transfected as transfection control. Cells were lysed 36 hours after transfection, and luciferase activity was measured using a Dual-Luciferase system (Promega, Madison, Wis.) according to the manufacture's protocol.

### (f) Invasion and Migration Assay

Transwell chambers (8-nm pore size, BD Falcon, Franklin Lakes, N.J.) were coated with the appropriate amount of Matrigel (BD Biosciences, San Jose, Calif.). Next, 2.5×10⁴ cells were suspended in NuSerum-containing media (Gibco BRL, Grand Island N.Y., USA), seeded in the chamber and cultured, Cells that invaded the chamber from top to bottom were fixed with methanol and stained with a solution of propidium iodine (Sigma-Aldrich, St. Louis, Mo.). The propidiumiodine-positive signal was quantified using Analytical Imaging Station software package. Each sample was assayed in triplicate.

For the migration assay, culture inserts (Ibidi, Munich, Germany) were inserted into 60-mm dishes. Next, the cell suspensions were seeded in each culture insert well at a concentration of 2.5×10⁴ (CL1-0) or 3×10⁴ (CL1-5-F4 and UACC-257) cells/mL. The culture inserts were removed to leave a gap of approximately 500 μm. Cell migration was observed at different time points, and the number of cells that migrated into the gap was calculated.

### (g) In Vivo Animal Models for Xenograft Tumors, Orthotopic Lung Tumors, and Metasitasis

Animals were housed in a specific-pathgen-free environment in the animal facility of the Institute of Biomedical Sciences, Academia Sinica. All experimental procedures were in compliance with the Academia Sinica IACUC and Council of Agriculture Guidebook for the Care and Use of Laboratory Animals. For intravenous injections of the tumor cells, 1×10⁶ cells were suspended in 0.1 mL of phosphate-buffered saline (PBS) and injected into lateral tail vein of SCID mice (10 mice per group). At 8 weeks after injection, all mice were killed, and lung surface tumor foci were counted. For the subcutaneous tumor assay, 1×10⁶ CL1-0 cells or 2×10⁵ H441 cells were subcutaneously injected in 0.1 mL of PBS into male nude mice (n=8 per group) and allowed to grow for 35 days to reach a volume of 50-200 mm³ Control antagomir (Scramble) or anti-miR-135b antagomir (antago-135) was intravenously injected at a concentration of 10 μM in 0.1 mL of PBS 4 days after the cells were implanted. An example of a miR-135b antagomir is 5′mU(*)mC(*)mAmCmAmUmAmG-mGmAmAmUmGmAmAmAmAmGmCmC(*)mA(*)mU(*)mA(*)(3′-Ch1)3′ (SEQ ID NO:8). The mN indicates 2′-O-methyl base;* indicates phosphorothioate linkage; Ch1 indicates cholesterol.

For the orthotopic tumor implantation assays, lentivirus-infected CL1-0/vector or CL1-0/miR-135b-overexpressiong cells (10⁵ Cells in 20 μL of PBS containing 10 ng of Matrigel) were injected into the pleural cavity of 6-week-old SCID mice (n=10 per group). The mice were killed by carbon dioxide anesthesia 28 days after implantation and the lungs were removed and fixed in 10% formalin. The lung nodules were counted by gross and microscopic examination. The number of mice used for experiments (n=10) was based on the goal of having 98% power to detect a twofold between-group in the number of modules at P<0.05.

### (h) Clinical Lung Cancer Samples and Immunohistochemistry

Frozen lung cancer specimens from 112 consecutive patients who underwent surgical resection of NSCLC (non-small-cell lung carcinoma) at Taichung Veterans General Hospital were analyzed for the expression of miR-135b (SEQ ID NO:9). None of the patients had received adjuvant chemotherapy, MicroRNA expression profiling was performed using a TaqMan MicroRNA Assay Kit (Applied Biosystems, Foster City, Calif.) and an ABI PRISM 7900 Real-Time PCR System. miR-135b expression was quantified in relation to the expression of small nuclear U6 RNA.

In addition, samples from 147 NSCLC patients who underwent surgical resection at the National Taiwan University Hospital were analyzed for the expressions of LZTS1, LATS2, and nuclear TAZ. Sections were fixed in formalin and embedded in paraffin. The primary antibodies against LZTS1 (anti-FEZ), LATS2, and TAZ were obtained from BD Biosciences (San Jose, Calif.), Bethyl Laboratories Inc., and Cell Signaling, respectively. PBS without primary antibodies was applied as the negative control. The immunohistochemistry results were scored and classified into 2 groups according to the average staining intensity and area. Group 1 corresponded to a positive staining of <50% of the tissue section, and group 2 corresponded to a positive staining of >50% of the tissue section. The immunostaining results were assessed and scored independently by two pathologists.

### (i) Chromatin Immunoprecipitation

CL1-0 and H1299 cells were fixed with 1% formaldehyde and blocked by 125 mM glycine. The cells were resuspended in cell lysis buffer (5 mM HEPES, 85 mM KCl, 0.5% Triton X-100, 1 mM DTT, 1 mM PMSF, pH 8.0), followed by nucleic lysis buffer (50 mMTris-HCl, 10 mM EDTA, 1% SDS, 1 mM DTT, and protease inhibitor (Roche Applied Science, Mannheim, Germany)). The cell lysate was sonicated and clarified by centrifugation. The supernatant was diluted with protein G beads at 4° C. to pre-clear the solution Immunoprecipitation with anti-RelA (Abcam, Cambridge, UK) was performed at 4° C. overnight. DNA-protein complexes were than incubated with protein G agarose beads at 4° C. with constant rotation for 2 hours. Following immunoprecipitation, the beads were washed with a low-salt wash buffer, a high-salt wash buffer, a LiCl wash buffer, and, finally, a TE buffer. The immunoprecipitated complexes were eluted in a buffer containing 10 mM Tris pH 8.0, 300 mM NaCl. 5 mM EDTA, and 0.5% SDS at room temperature. The samples were then treated with proteinase K for 1 hour, followed by RNAse A. Next, the DNA was purified by phenol/chloroform extraction. The DNA was submitted for PCR amplification with primers specific to the miR-135b promoter region: the forward primer was SEQ ID NO: 10, and the reverse primer was SEQ ID NO: 11.

### (j) Statistical Analysis

Data are presented as the mean±s.d. The difference between two groups were assessed using the Student's t-test, and the Kaplan-Meier survival analysis was used to estimate overall survival. Differences in survival between two groups were analyzed using the log-rank test. Multivariate Cox proportional hazard regression analysis with stepwise selection was used to evaluate independent prognostic factors associated with patient survival, and the expression of miR-135b, age, gender, tumor stage, and histology were used as covariates. All analyses were performed with SAS version 9.1 software (SAS Institute Inc.). Two-tailed tests were used, and P value <0.05 were considered to indicate statistical significance.

### 2. Results

### Example 1

### Identification of Invasion-Associated miRNAs in Lung Cancer Cells

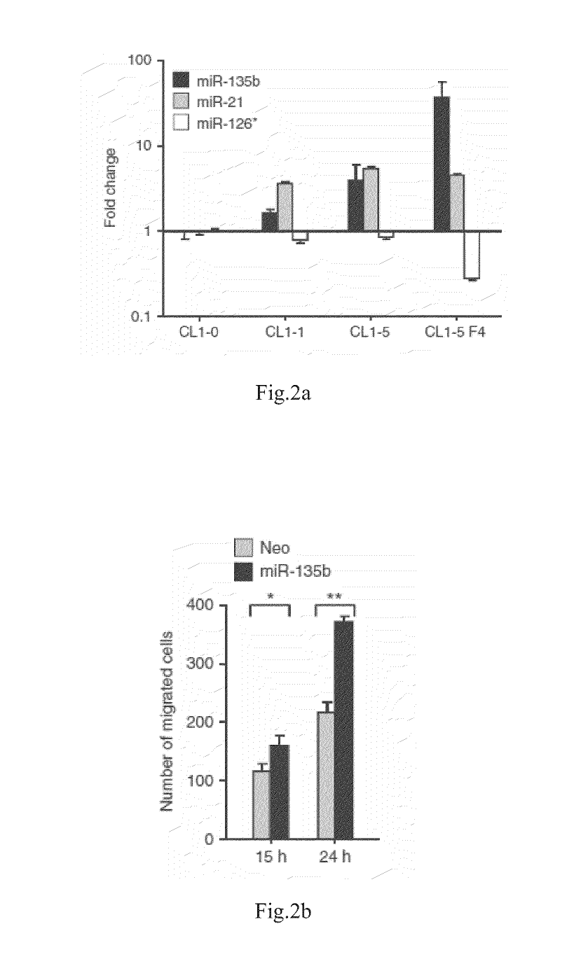

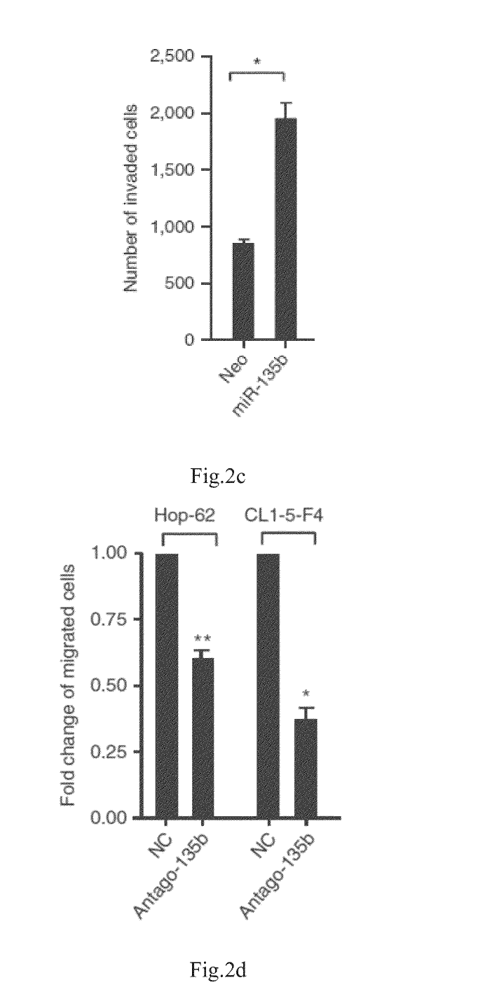

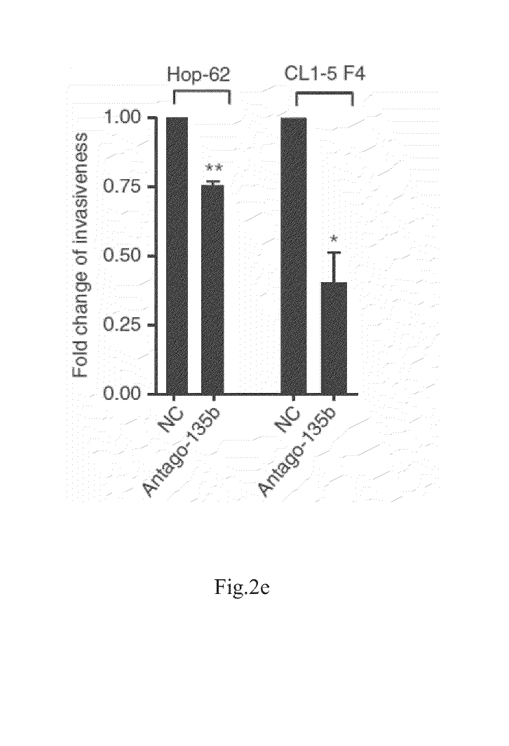

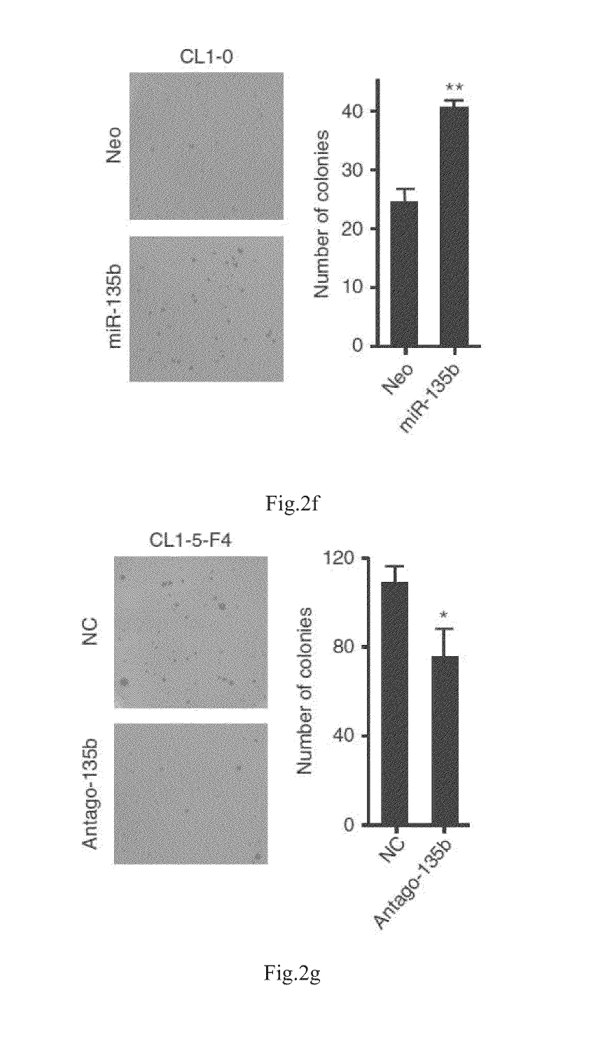

To identify invasion-associated miRNAs in lung cancer cells, a miRNA microarray was conducted in lung cancer cell sub-lines of increasing invasive potential. Several miRNAs were found differentially expressed in these cell lines (Table 1). For example, the expression of oncomiR miR-21 was increased by approximately twofold in highly invasive CL1-5 compared to the less invasive CL1-0 cells. In contrast, the expression of miR-126/126*, which is associated with a tumor suppressor function in invasive lung cancer cells, was decreased in CL1-5 cells. The greatest elevations of miR-135b levels were found in highly invasive CL1-5 cells (Table 1). As shown in FIG. 2a, real-time RT-PCR confirmed the expressions of miR-21, miR-126. and miR-135b in CL-series cells lines. In concordance with these results, miR-135b expression was positively correlated with increasing invasive activities of these lung cancer cell lines.

TABLE 1
Selected miRNA microarray results showing differential expressions
of miRNAs in less invasive CL1-0 and highly invasive CL1-5 cells.

| | CL1-0 | CL1-5 | CL1-5/CL1-0 |
|---|---|---|---|
| miR-135b | 149 | 1225 | 8.23 |
| miR-135b | 150 | 1679 | 11.2 |
| miR-21 | 702 | 1612 | 2.29 |
| miR-21 | 580 | 1134 | 1.96 |
| miR-126 | 208 | 122 | 0.59 |
| miR-126 | 212 | 111 | 0.52 |
| miR-126* | 175 | 119 | 0.68 |
| miR-126* | 180 | 91 | 0.50 |

### Example 2

### miR-135b Promotes Cancer Cell Growth and EMT (Epithelial-Mesenchymal Transition) In Vitro

The effects of miR-135b on cell invasion and migration were evaluated. A pri-miR-135b lentiviral expression vector was used to induce miR-135b expression in CL1-0 cells, and miRNA levels were assayed using real time RT-PCR. As shown in FIG. 2b and FIG. 2c, the ectopic expression of miR-135b in CL1-0 cells significantly increased migratory and invasive abilities. As shown in FIG. 2d and FIG. 2e, the inhibition of miR-135b by antagomiR and antisense oligonucleotides inhibited these changes in highly invasive CL1-5-F4 cells and in Hop-62 cells. Moreover, miR-135b promoted EMT in CL1-0 and HEK-293 cells, and inhibition of miR-135b altered the expression of E-cadherin in CL1-5 and CL-141 cells. Thus, the results of all of these experiments confirmed that miR-135b could promote cancer cell migration and invasion.

As shown in FIG. 2f, to address other oncogenic activities modulated by miR-135b, an anchorage-independent assay was performed. Overexpression of miR-135b promoted anchorage-independent growth CL1-0 cells. Conversely, as shown in FIG. 2g, miR-135b antagomir decreased the number of CL1-5-F4 cell colonies, suggesting that miR-135b governs both invasiveness and anchorage-independent growth in lung caner cells.

### Example 3

### miRNA-135b Promotes Tumor Growth and Metastasis In Vivo

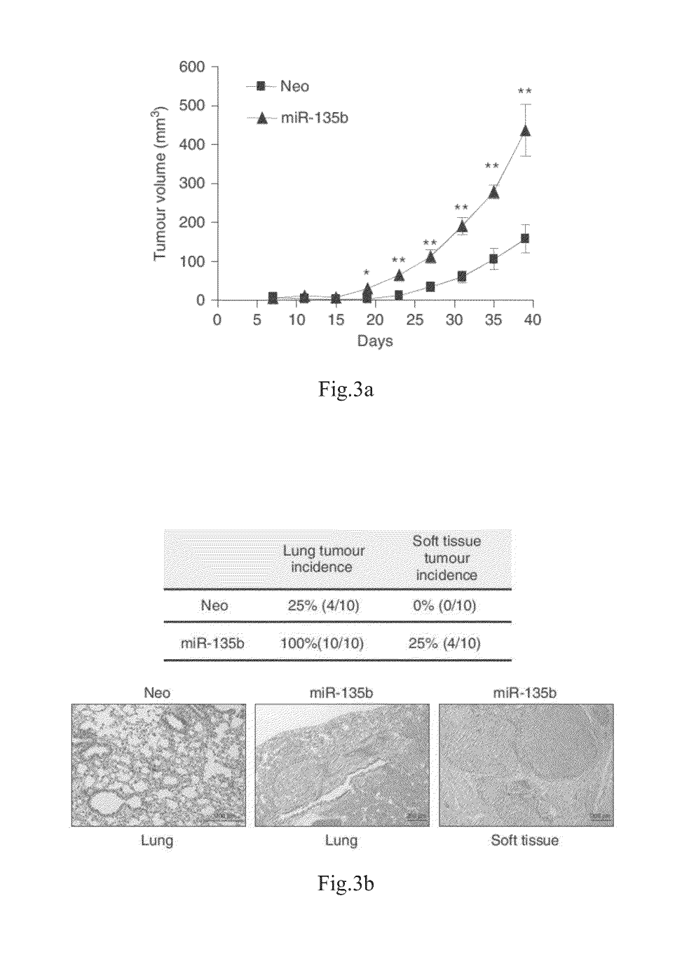

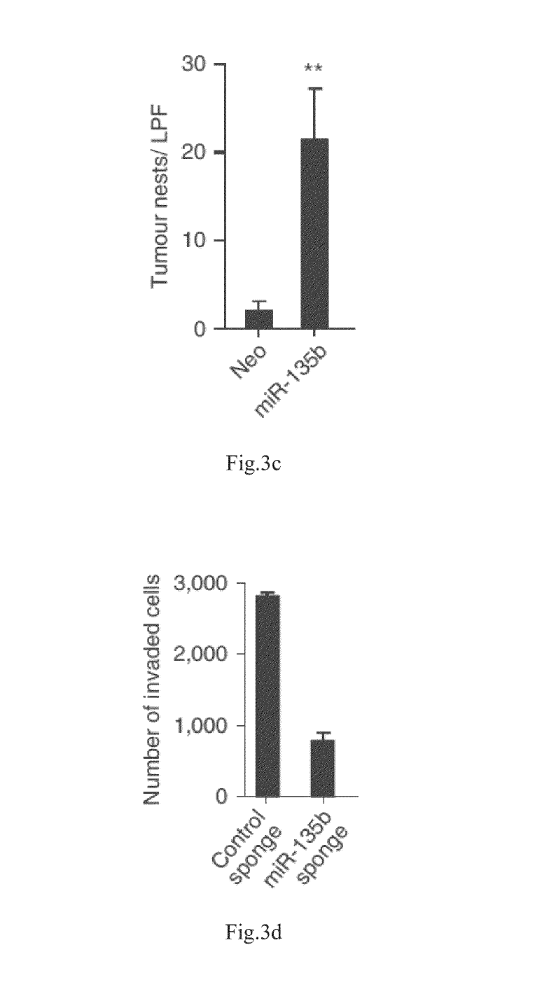

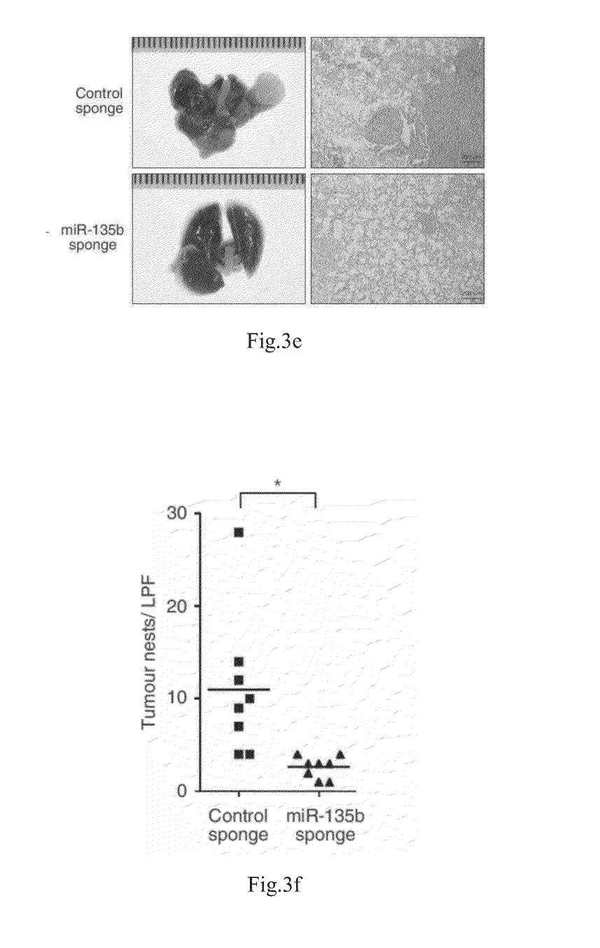

To evaluate the effect of miR-135b on tumor growth in vivo, the expression level of miR-135b in CL1-0 cells was manipulated and then subcutaneous xenograft of these cells was performed into nude mice. As shown in FIG. 3a, CL1-0 cells overexpressing miR-135b exhibited tumorigenic ability 20 days after implantation.

To test the effects of miR-135b on in vivo cell metastasis, as shown in FIGS. 3b and 3c, CL1-0 cells were stably transduced with a miR-135b-expressing lentiviral vector. NOD-SCID mice were intravenously injected with CL1-0 cells and sacrificed after 8 weeks. miR-135b expression promoted metastasis in lung and soft tissues. Furthermore, whether suppression of miR-135b expression would impede lung cancer invasion and metastasis was investigated. A miR-135-specific sponge carrying seven repeats of the miR-135b binding site was constructed to neutralize endogenous miR-135b activity. As shown in FIG. 3d, highly invasive CL1-5-F4 cells stably expressing either a control or the miR-135b sponge (SEQ ID NO:12) were analyzed by transwell invasion assay, and cells expressing the miR-135b sponge diminished the invasive. Next, as shown in FIG. 3e, the SCID mice were intravenously injected with control or miR-135b sponge-producing CL1-5-F4 cells and the number of metastatic lung nodules 6 weeks later was counted. In FIG. 3f, the number of tumor nodules that developed in the mouse lungs was significantly reduced in the miR-135b sponge group (P=0.0126). The overexpression of miR-135b in highly invasive cells appeared to be required for in vivo lung cancer growth and metastasis in these experiments.

### Example 4

### miR-135b Antagomir Inhibits Lung Tumor Growth and Metastasis

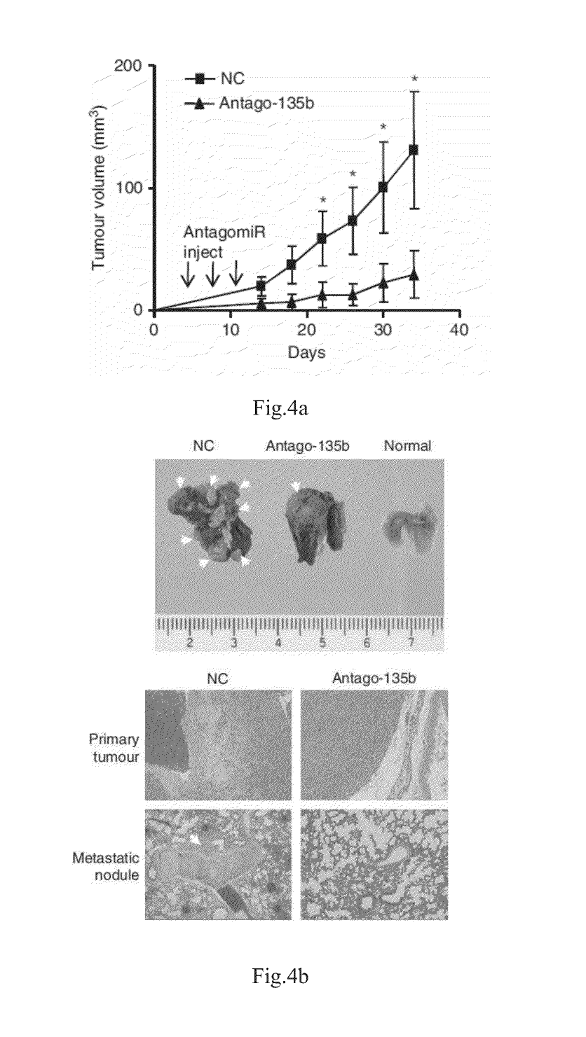

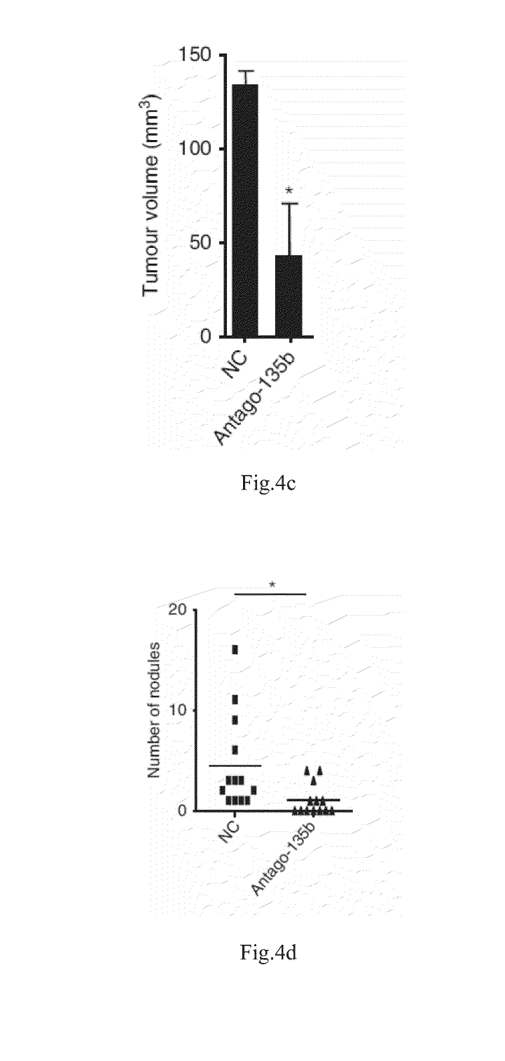

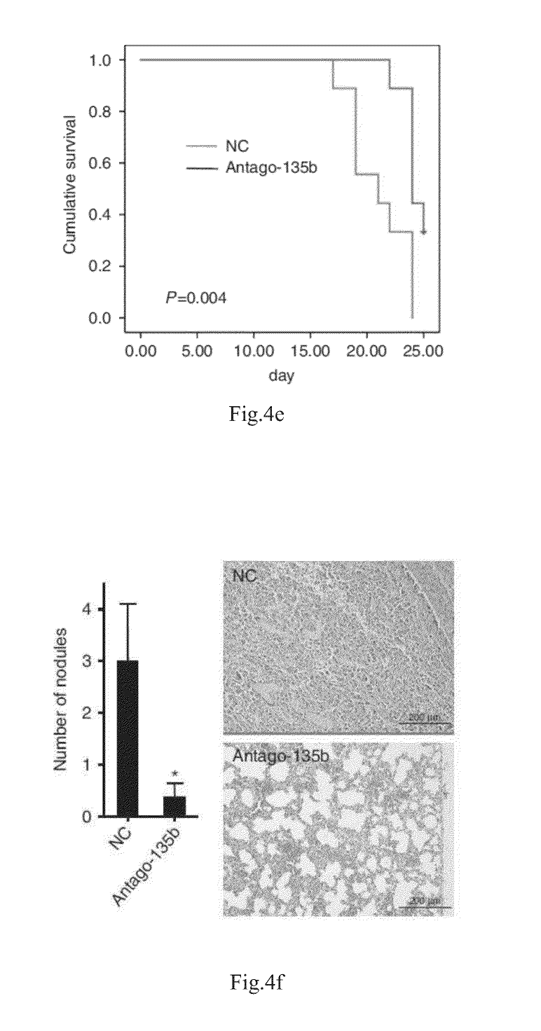

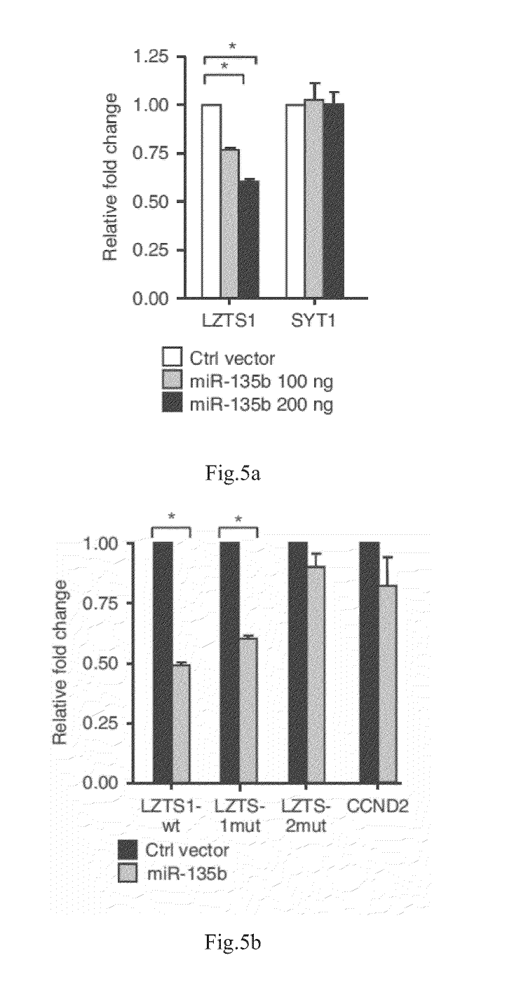

The therapeutic potential of miR-135b antagomir in three sets of animal models was examined. First, the effects of the inhibition of endogenous miR-135b on tumor growth in xenograft tumors were tested. The growth of H441 human lung adenocarcinoma cells, which express high levels of endogenous miR-135b, was inhibited when anatgo-135b antagomir was administered before xenograft implantation. As shown in FIG. 4a, this inhibitory activity was sustained when miR-135b antagomir was systematically injected 4 days after cancer cell implantation, and the incidence and volume were significantly reduced in the xenograft tumors.

Next, the effects of miR-135b inhibition on tumor growth and metastasis in an orthotopic lung cancer model were checked. Seven intravenous injections of miR-135b antagomir were given 4 days after CL1-5 implantation. Compared with the control lungs, miR-135b antagomir inhibited orthotopic tumor growth (FIG. 4b and FIG. 4c) and decrease the volume of lung metastases (FIG. 4b and FIG. 4d). In addition, as shown in FIG. 4e, even though the severe growth of orthotopic tumors caused cachexia and death of the mice, systemic delivery of the antago-135b antagomir in the tumor-bearing mice increased survival rates.

The impact of the antagomir during the late stages in metastasis was determined, such as extravasation and colonization. Intravenous injections of highly invasive CL1-5-F4 cells in the mice were followed by a succession of either control- or antago-135b treatments via the ail vein. As shown in FIG. 4f, on evaluation 3 months after the cells were injected, the antago-135b-treated group had generated eight-fold fewer metastatic lung nodules than the control group (P=0.018). Thus, with mi-RNA-135b as a therapeutic target, the administration of antago-135 antagomir controlled lung cancer growth and metastasis in the experimental mouse models.

### Example 5

### Mi-RNA-135b Regulates LZTS1 to Control Cancer Invasion

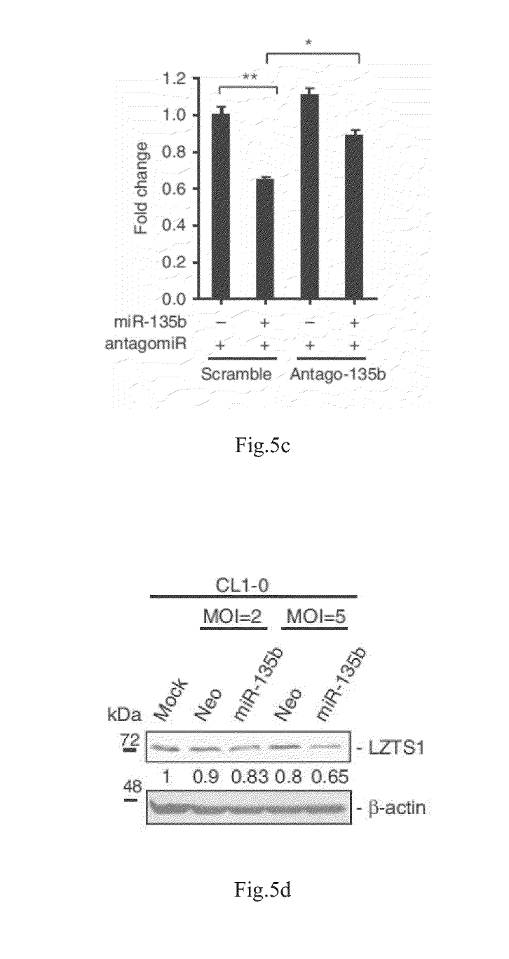

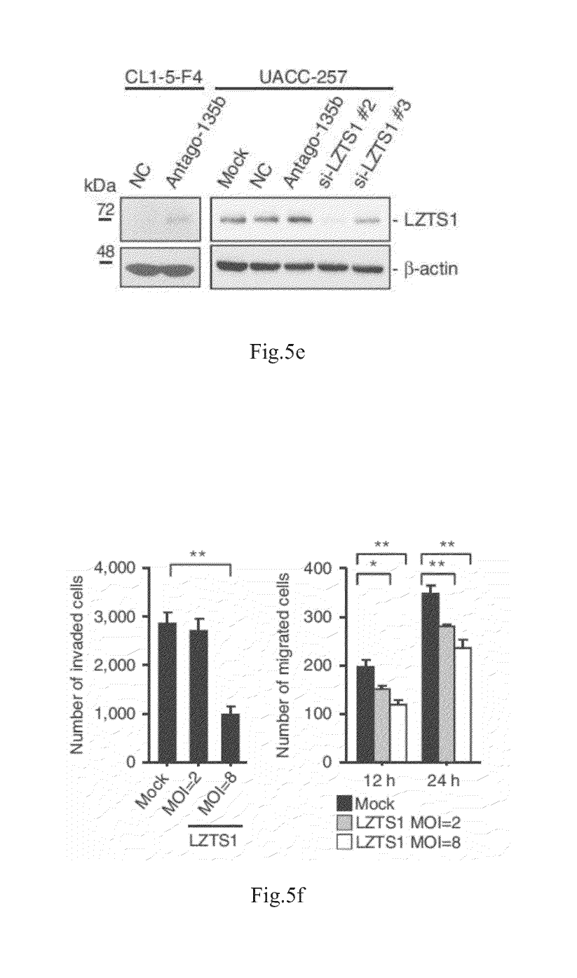

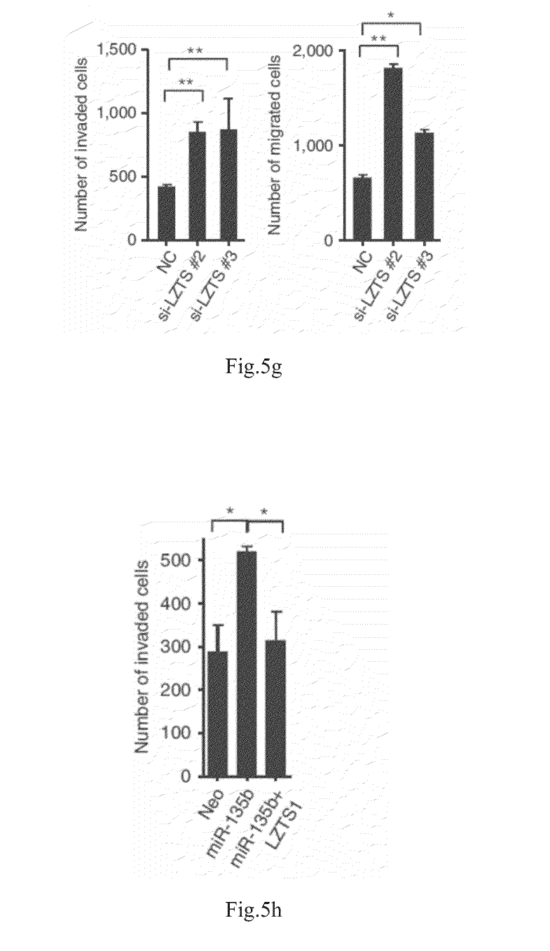

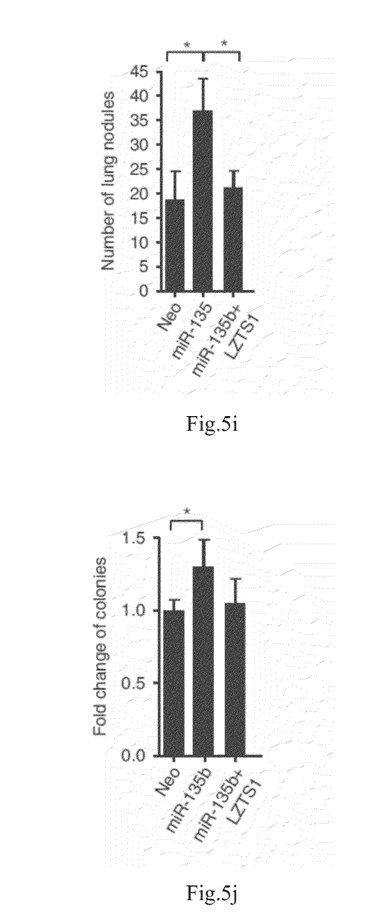

Target genes of miR-135b were identified using the computational algorithms of TargetScan (Version 5.2) for prediction anaylsis. Several candidates were discovered, and their 3′-UTRs (3′-untranslated regions) were conjugated with luciferase for reporter assays. LZTS1 has the potential to suppress the invasion and motility of melanoma cells, and its expression is associated with lymph node metastasis in breast cancer patients. In the present invention, as shown in FIG. 5a, the luciferase activity of plasmids containing the 3′-UTR of LZTS1 (SEQ ID NO:13) was significantly reduced in the presence of miR-135b. The putative miR-135b seed sequence mutations were introduced to further investigate the direct regulatory effect. As shown in FIG. 5b, the reporter assay showed that the effects of miR-135b repression were abolished when both putative seed sequence were mutated. As shown in FIG. 5c, the suppressive effects of miR-135b on the LZTS1 3′-UTR-carrying luciferase were significantly reduced by the antago-135 antagomir. These results indicated that miR-135b regulated the expression of LZTS1 through a direct seed sequence interaction.

Next, the luciferase gene was replaced by the LZTS1 coding sequence to mimic the endogenous LZTS1 transcript. The overexpression of miR-135b decreased the expression of HA-tagged LZTS1 protein in a dose-dependent manner. The miR-135b-mediated suppression was negated by mutation of the miR-135b seed sequences on the LZTS1 3′-UTR.

To investigate whether miR-135b regulates endogenous LZTS1, the expression of LZTS1 in miR-135b-expressing lentiviral vector-transduced cells was evaluated. As shown in FIG. 5d, endogenous LZTS1 expression in the lung cancer cell line CL1-0 was decreased by the ectopic expression of miR-135b. The same phenomenon was observed in UACC-257 melanoma cells and in MDAMB-435 cells. In contrast, as shown in FIG. 5e, blocking of miR-135b with antisense oligonucleotides significantly increased LZTS1 expression in CL1-5-F4 and USCC-257 cells. These results demonstrated that miR-135b-mediated LZTS1 repression is possible in lung cancer cells and in other types of cancer cells.

To explore the biological function of LZTS1 in lung caner cells, a lentiviral vector containing the complete coding sequence of LZTS1 was transduced into A549 cells. As shown in FIG. 5f, the ectopic expression of LZTS1 suppressed cell invasion and migration in a dose-dependent manner, as shown in FIG. 5g, while suppression of LZTS1 expression by double-strand siRNA in UACC-257 cells enhanced cell invasion and migration. This resembled the effects of miR-135b overexpression in low-invasive cells. Conversely, as shown in FIG. 4h, the invasive activity was reduced if miR-135b was overexpressed in conjunction with LZTS1 in CL1-0 cells. As shown in FIG. 5i, the in vivo metastatic assay also showed that LZTS1 remarkably suppressed the lung metastatic nodules and was sufficient to repress miR-135b-dependent metastatic colonization and Slug protein expression. However, as shown in FIG. 5j, an overexpression of LZTS1 in miR-135b expression cells did not repress the miR-135b-driven colony forming activity. These results suggest that major function of the miR-135b-LZTS1 axis in lung cancer is the suppression of cancer metastasis.

### Example 6

### miRNA-135b Regulates Multiple Components of Hippo Pathway

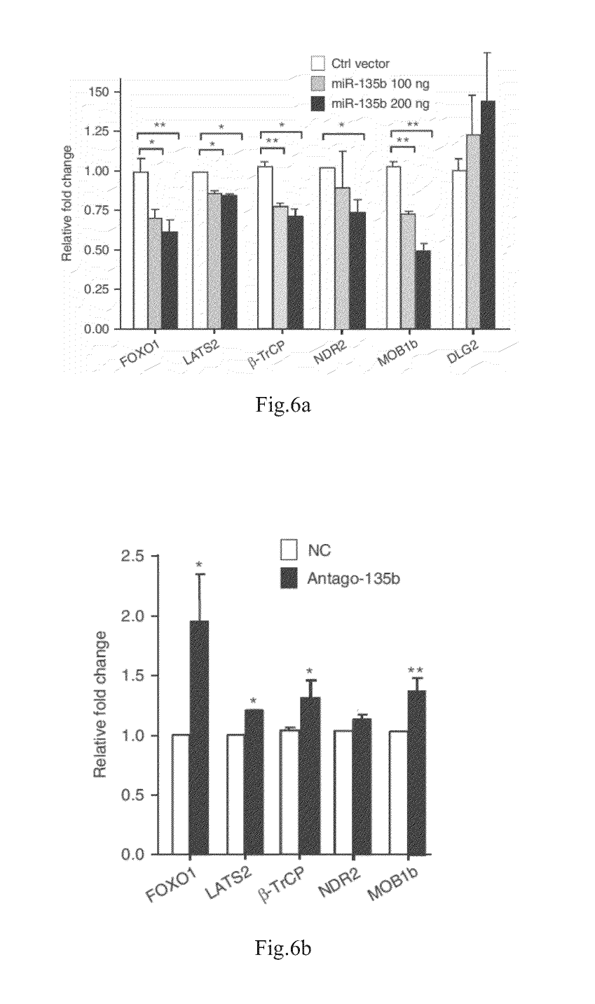

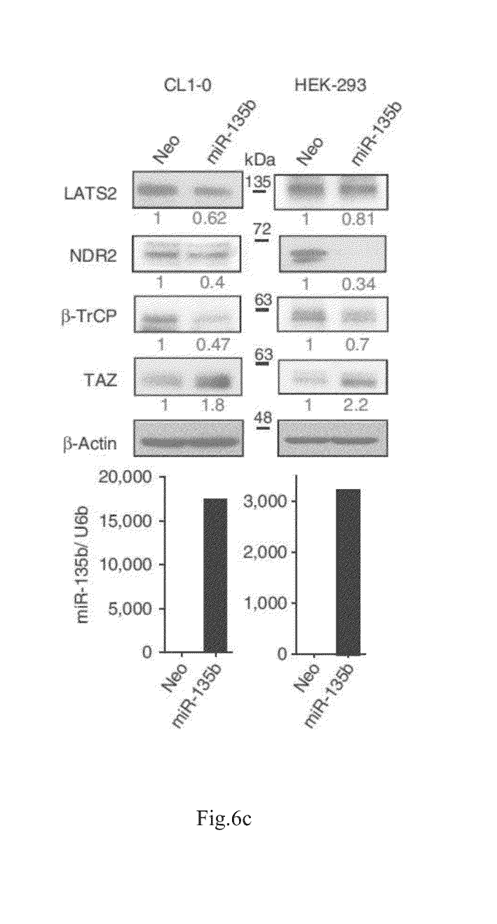

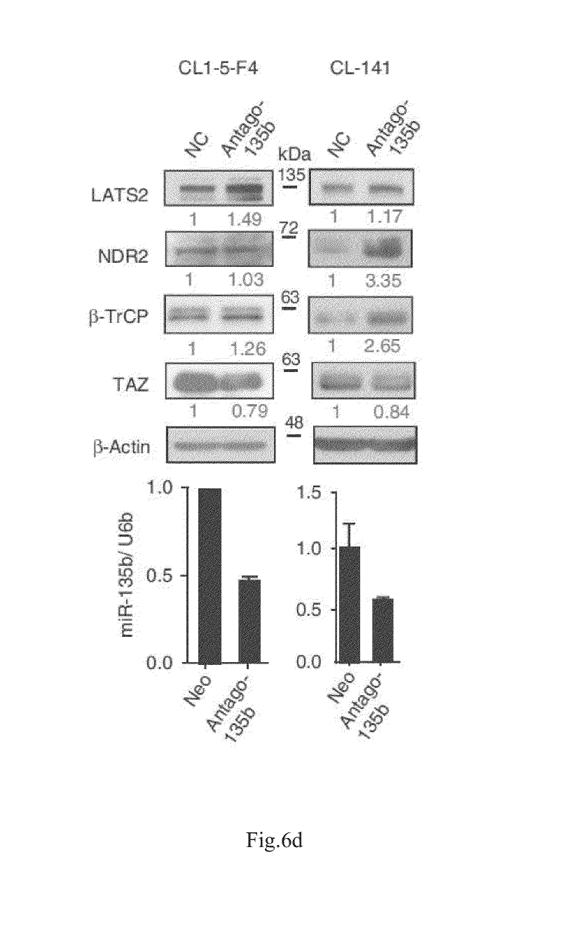

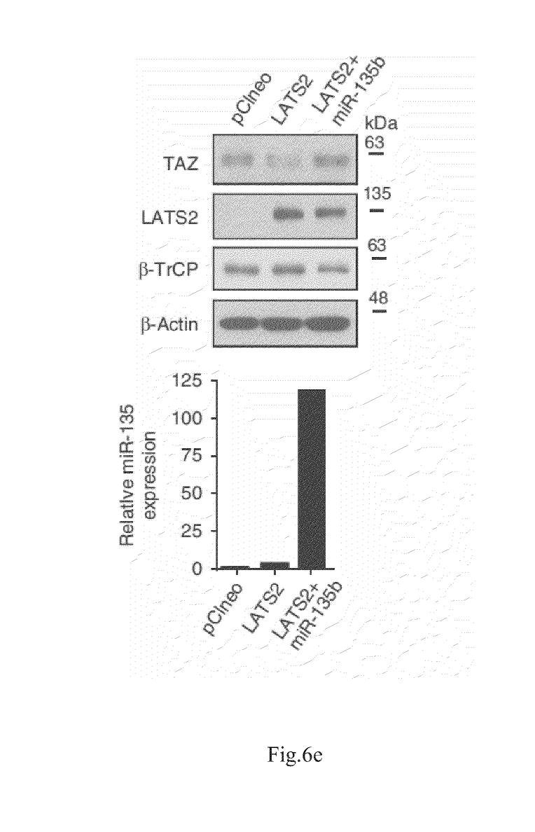

The Hippo pathway plays an important role in controlling organ size in Drosophila melanogaster and tumorigenesis in mammals. The central axis of the Hippo pathway is a kinase cascade that include MST1, LATS1/2 (serine/threonine-protein 1/2), and MOB1a/b (Mob kinase activator 1a/b), along with downstream TAZ oncogenic effectors. Phosphorylaion of TAZ is initiated at Ser 311, and CK1 phosphorylates Ser 314, which leads to a SCF^{β-TrCP} (beta-transducin repeat-containing protein)-mediated ubiquitination and degradation. NDR1/2 (nuclear Dbf2-related kinase 1/2) and FOXO1 are also phosphorylated by MST1 when the Hippo pathway is activated, and this is thought to assist with the tumor suppressive function of MST1. Based on a TargetScan (Version 5.2) prediction, six Hippo pathway-related genes were identified to contain putative miR-135b target sites on their 3′UTRs. In agreement with this, a negative correlation between endogenous LATS2 and TAZ expression in CL-series cells was observed. As sown in FIG. 6a, the results of a luciferase reporter assay showed that miR-135b could downregulate the canonical Hippo pathway protein LATS2 as well parallel molecules including β-TrCP, NDR2 and MOB1b. As shown in FIG. 6b, administration of the antago-135b antagomir resulted in a decrease in miR-135b-induced reporter activity. As shown in FIG. 6c, endogenous protein levels of LATS2 (SEQ ID NO:14), β-TrCP and NDR2 were downregulated in miR-135b-expressing CL1-0 and HEK-293 cells. Conversely. TAZ, a major Hippo downstream effector, was upregulated in the miR-135b-overexpressing cells.

The regulation of the Hippo pathway was confirmed by miR-135b by treatment of CL1-5-F4 and CL141 cells with antago-135b antagomir. As shown in FIG. 6d, inhibition of miR-135b reduced TAZ protein expression and induced LATS2, 3-TrCP and NDR2 expressions, and endogenous TAZ was downregulated in miR-135b suppressed lung cancer cell lines. To further determine whether the canonical Hippo components were epistatically regulated by miR-135b, as shown in FIG. 6e, the function of miR-135b and the Hippo downstream effector was explored. And TAZ Transient expression of LATS2 in CL1-5 cells decreased the TAZ protein level. However, when miR-135b was introduced the endogenous TAZ was recovered, indicating that the expression of TAZ was associated with miR-135b. To identify the functions of the Hippo component, LATS2, and its downstream effector TAZ, although ectopic LATS2 did not affect cancer cell migration, invasion and colony-forming activity, knockdown of TAZ dramatically reduced cancer cell invasive and colony forming abilities. These finding suggest that miR-135b contributes to the oncogenic activation of TAZ via multiple in a Hippo targets in a Hippo pathway.

### Example 7

### Clinical Correlations of miR-135b and its Targets in NSCLC

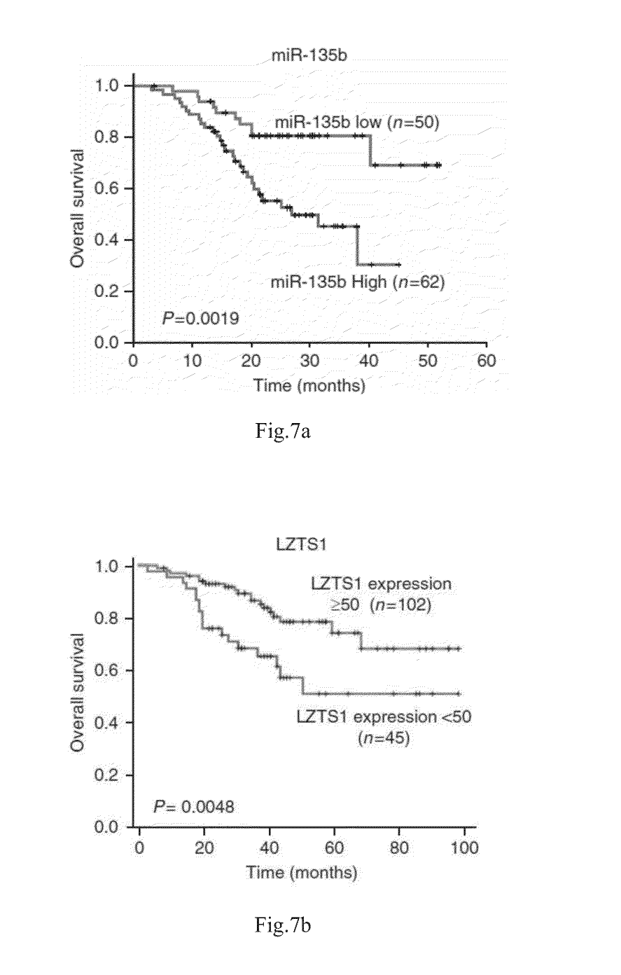

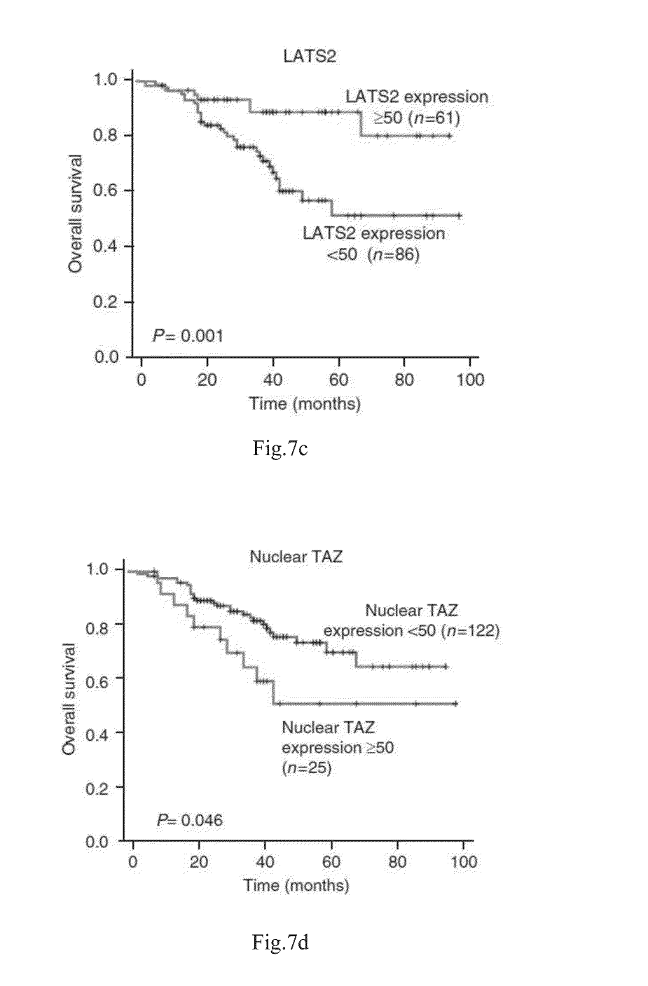

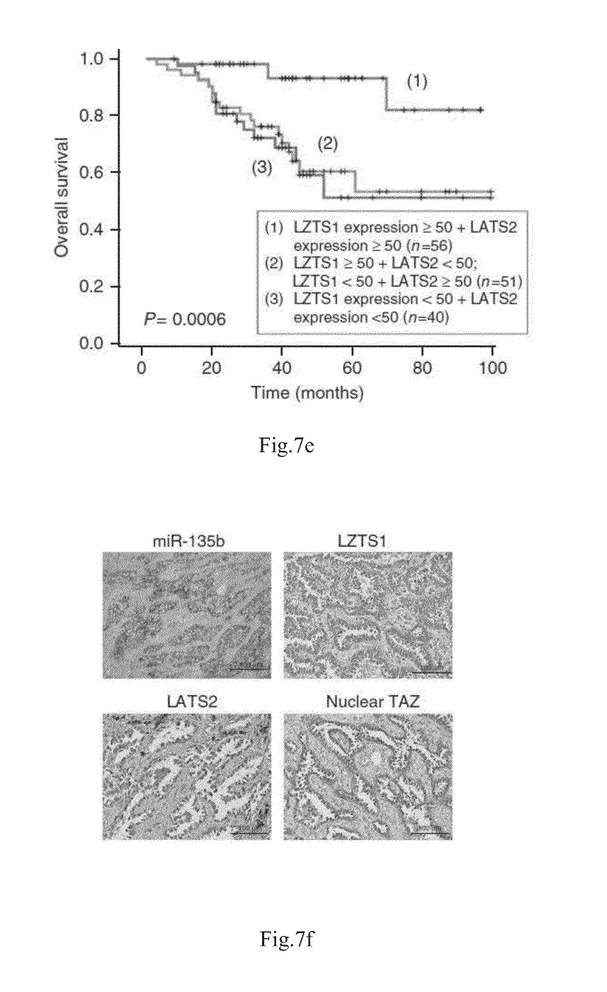

To further understand the potential biological significance of deregulated miR-135b expression in lung cancer progression, the correlation of the miR-135b expression profile with overall survival in tumor specimens from 112 lung cancer patients was evaluated (Table 2). As shown in FIG. 7a, miR-135b levels were measured by real time RT-PCR, and Kaplan-Meler analysis showed that high levels of miR-135b expression were significantly associated with decreased overall survival (P=0.0019). Cox proportional hazard regression analysis with a stepwise selection model also demonstrated that the overall survival of this cohort was correlated with the miR-135b expression levels (HR=2.24)(Table 3).

TABLE 2
Clinical characteristics of 112 NSCLC patients
evaluated for lung tumor miR-135b expression

| | High miR135b | Low miR135b | |
|---|---|---|---|
| Characteristics | Expression | Expression | P value |
| Age (mean ± SD) | 65.0 ± 12.0 | 65.1 ± 12.1 | 0.9773† |
| Gender | Patient no. (%) | Patient no. (%) | |
| Male | 39 (78.0%) | 46 (74.2%) | 0.6642* |
| Female | 11 (22.0%) | 16 (25.8%) | |
| Stage | | | |
| I | 23 (46%) | 24 (38.7%) | 0.0674* |
| II | 16 (32.0%) | 12 (19.4%) | |
| III | 11 (22.0%) | 26 (41.9%) | |
| Histology | | | |
| Adenocarcinoma | 17 (34.0%) | 38 (61.3%) | 0.0152* |
| Squamous cell | 29 (58.0%) | 21 (33.9%) | |
| carcinoma | | | |
| Others | 4 (8.0%) | 3 (4.8%) | |

†Student's t-test
*Fisher's exact test

TABLE 3
Mutivariate Cox regression* analysis of miR-135b levels and
overall survival in 112 NSCLC patients

| Variable | Hazard ratio (95% C.I.) | P value |
|---|---|---|
| Age | 1.04 (1.00~1.07) | 0.026 |
| Stage | 3.65 (1.86~7.13) | 0.002 |
| miR-135b | 2.24 (1.05~4.78) | 0.036 |

*Variables were selected through the stepwise selection method

Next, the expression of the miR-135b downstream target genes was examined, LZTS1 and LATS2, as well as nuclear TAZ, by immunohistochemical analysis of 147 NSCLC tumor samples (Table 4). As shown in FIG. 7b, Kaplan-Meier and log-rank test analyses demonstrated that lower LZTS1 expressions were associated with poor overall survival (P=0.048). In addition, inverse correlations with miR-135b expression levels by in situ hybridization and LZTS1 protein levels by immunohistochemistry in the same tumor sections were observed. As shown in FIG. 7c, tumor specimens with a low expression of LATS2 were associated with poorer overall survival (P=0.0015). Multivariate Cox analysis demonstrated that both LZTS1 (HR=0.494, 95% CI=0.251 to 0.971; P=0.0409) and LATS2 (HR=0.416, 95% CI=0.176 to 0.983; P=0.0455) were protective factors when age, gender, and tumor histological type were considered (Table 5 and Table 6), which suggests that patients with tumors expressing higher levels of LZTS1 and LATS2 may have a lower risk of mortality. In addition, nuclear TAZ was found to be a risk factor for survival outcome (HR=3.079, 95% CI=1.409 to 6.727; P=0.0048). As shown in FIG. 7d, high levels of nuclear TAZ staining, which indicated TAZ activation in the tumor specimens, were associated with poor overall survival in the patients (P=0.049).

TABLE 4
Clinical characteristics of 147 NSCLC patients with different
expression levels of LZTS-1, LATS-2, and nuclear TAZ in tumor specimens*

| | | | | | | | | Nuclear | Nuclear | |
|---|---|---|---|---|---|---|---|---|---|---|
| | No. of | LZTS-1 | LZTS-1 | P | LATS-2 | LATS-2 | P | TAZ | TAZ | P |
| Parameter | Patients | <50% | ≧50% | value | <50% | ≧50% | value | <50% | ≧50% | value |
| Number of Patients (%) | 147 | 45 (30.6) | 102 (69.4) | | 86 (58.5) | 61 (41.5) | | 122 (83.0) | 25 (17.0) | |
| Age (mean ± SD) | | 64.6 ± 10.6 | 63.5 ± 10.8 | 0.571 | 63.9 ± 10.6 | 63.7 ± 10.9 | 0.920 | 64.4 ± 10.5 | 60.9 ± 11.5 | 0.134 |
| Sex | | | | | | | | | | |
| Male | 67 | 20 (29.9) | 47 (70.2) | 0.855 | 40 (59.7) | 27 (40.3) | 0.787 | 59 (88.1) | 8 (11.9) | 0.135 |
| Female | 80 | 25 (3.3) | 55 (68.8) | | 46 (57.5) | 34 (42.5) | | 63 (78.8) | 17 (21.3) | |
| Histological type† | | | | | | | | | | |
| Squamous ce8 | 26 | 8 (30.8) | 18 (69.2) | 0.985 | 19 (73.1) | 7 (26.9) | 0.096 | 19 (73.1) | 7 (26.9) | 0.136 |
| carcinoma | | | | | | | | | | |
| Adenocarcinoma | 121 | 37 (30.6) | 84 (69.4) | | 67 (55.4) | 54 (44.6) | | 103 (85.1) | 18 (14.9) | |
| Tumor size, cm | | | | | | | | | | |
| ≦3 | 58 | 19 (27.9) | 49 (72.1) | 0.515 | 37 (54.4) | 31 (45.6) | 0.350 | 54 (79.4) | 14 (20.5) | 0.284 |
| >3 | 79 | 26 (32.9) | 53 (67.1) | | 49 (62.0) | 30 (38.0) | | 68 (86.1) | 11 (13.9) | |
| Tumor stage | | | | | | | | | | |
| Stage I-II | 124 | 34 (27.4) | 90 (72.6) | 0.061 | 68 (54.3) | 56 (45.2) | 0.036 | 102 (82.3) | 22 (17.7) | 0.582 |
| Stage III-IV | 23 | 11 (47.8) | 12 (52.2) | | 18 (78.3) | 5 (21.7) | | 20 (87.0) | 3 (13.0) | |
| LZTS-1 expression | | | | | | | | | | |
| <50% | 45 | — | — | — | 40 (86.9) | 5 (11.1) | <0.001 | 36 (60.0) | 9 (29.0) | 0.521 |
| ≧50% | 102 | — | — | | 45 (45.1) | 66 (54.9) | | 86 (84.3) | 15 (15.7) | |
| LATS-2 expression | | | | | | | | | | |
| <50% | 86 | 40 (46.5) | 46 (53.5) | <0.001 | — | — | — | 70 (81.4) | 16 (18.6) | 0.540 |
| ≧50% | 61 | 5 (8.2) | 56 (91.8) | | — | — | | 52 (85.3) | 9 (14.8) | |
| Nuclear TAZ | | | | | | | | | | |
| expression | | | | | | | | | | |
| <50% | 122 | 36 (29.5) | 86 (70.5) | 0.521 | 70 (57.4) | 52 (42.6) | 0.540 | — | — | — |
| ≧50% | 25 | 9 (36.0) | 16 (64.0) | | 16 (64.0) | 9 (36.0) | | — | — | |

*P values were calculated using a two-sided chi-squared test
†Adenosquamous carcinoma was not included in the histological group

TABLE 5
Hazard ratios among NSCLC patients with LZTS1 or LATS2
expression according to multivariate Cox regression analysis*

| Variable | Hazard ratio (95% CI) | P value |
|---|---|---|
| Age | 0.977 (0.946~1.008) | 0.141 |
| Histological type | 0.501 (0.231~1.090) | 0.0815 |
| Sex | 1.510 (0.741~3.079) | 0.2563 |
| LZTS1 | 0.494 (0.251~0.971) | 0.0409 |
| LATS2 | 0.416 (0.176~0.983) | 0.0455 |

*Stepwise selection was used to choose the optimal multivariate Cox proportional hazard regression model. LZTS1 and LATS2 expressions were designated as “high” or “low” using 50% cell positivity as the cutoff point, and this was adjusted by age, histological type (squamous cell carcinoma as the referent vs. adenocarcinoma), and gender (male vs. female). P values (two-sided) were calculated using a chi-square test.
CI, confidence interval.

TABLE 6
Hazard ratios among NSCLC patients with tumor expressing both
LZTS1 or LATS2 according to multivariate Cox regression analysis*

| Variable | Hazard ratio (95% CI) | P value |
|---|---|---|
| Age | 0.978 (0.948~1.01) | 0.1699 |
| Histological type | 0.533 (0.244~1.164) | 0.1142 |
| Sex | 1.506 (0.738~3.074) | 0.2610 |
| LZTS1 and LATS2 | 0.575 (0.368~0.898) | 0.0149 |

*Stepwise selection was used to choose the optimal multivariate Cox proportional hazard regression model. LZTS1 and LATS2 co-expressions were designated as “both high” or “both low” using 50% cell positivity as the cutoff point, and this was adjusted by age, histological type (squamous cell carcinoma as the referent vs. adenocarcinoma), and gender (male vs. female). P values (two-sided) were calculated using a chi-square test.
CI, confidence interval.

The combined effects of both proteins on the prognosis of NSCLC patients were further analyzed, as shown in FIG. 7e, the patients with tumors expressing higher levels of LZTS1 and LATS2 had better overall survival than those whose tumor showed low LZTS1 and LATS2 expressions (P=0.0006). Multivariate Cox analysis also showed that these two proteins were protective factors if both were expressed at higher levels (HR=0.575, 95% CI=0.368 to 0.898; P=0.0149). These results indicated that the survival of NSCLC patients strongly associated with miR-135b and its LZTS1 and LATS2 targets. FIG. 7f shows a typical in situ hybridization signal of miR-135b and immunohistochemical staining of LZTS1, LATS2, and nuclear TAZ. The expressions of miR-135b, LZTS1, LATS2, and nuclear TAZ can be a useful prognostic signature for NSCLC.

### Example 8

### Dual Transcriptional Regulation of miR-135b Expression

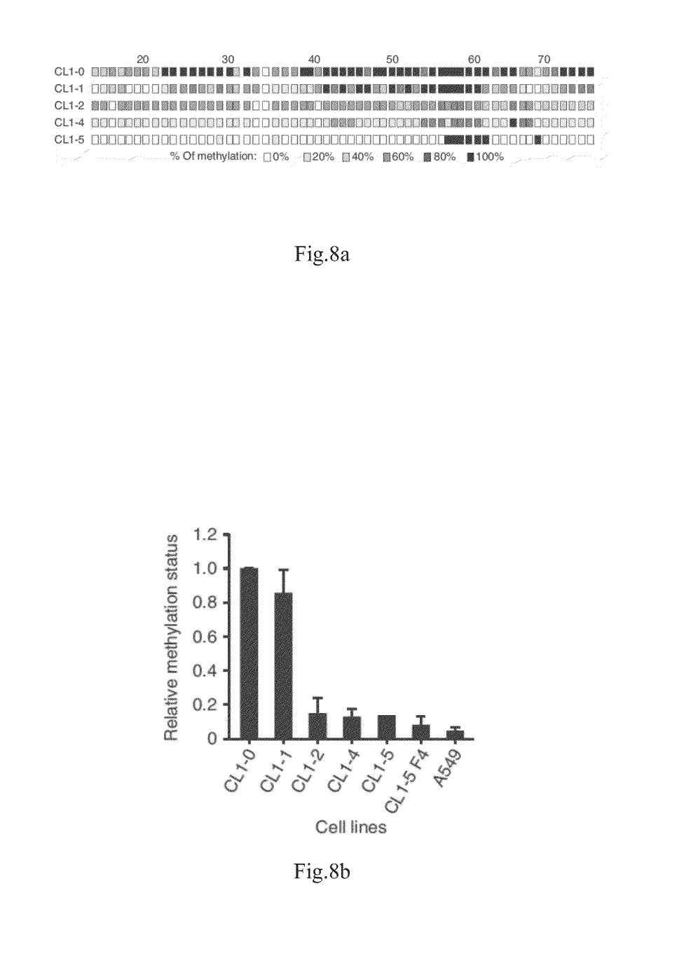

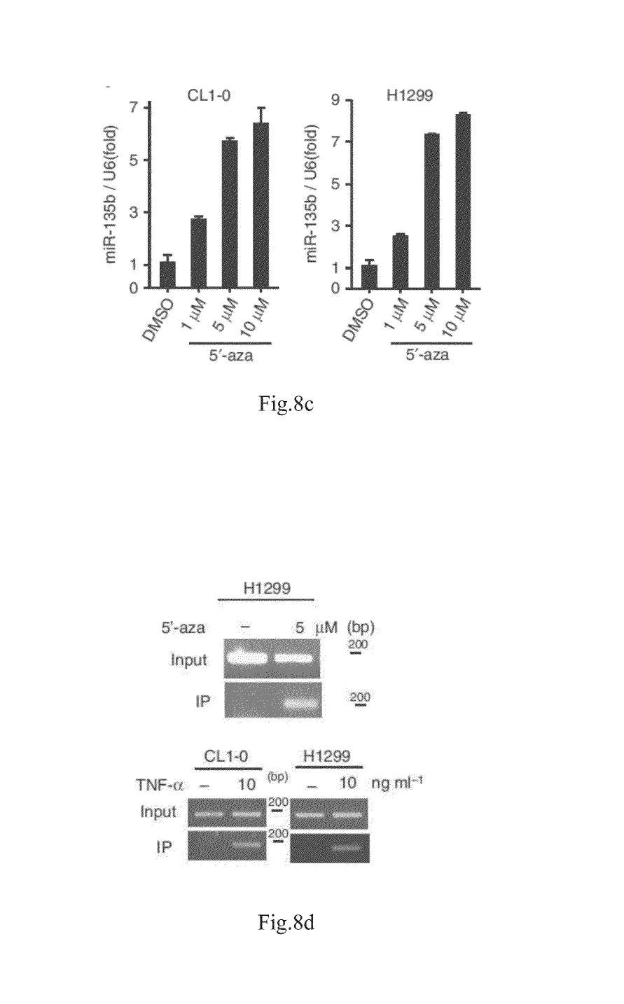

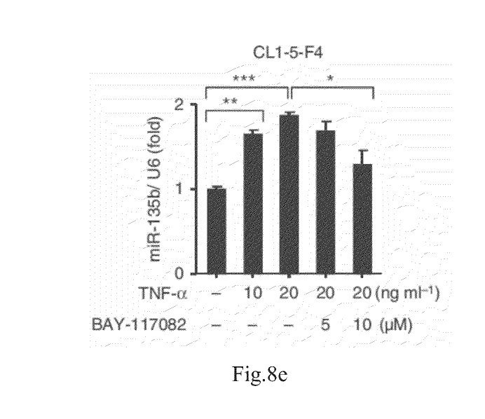

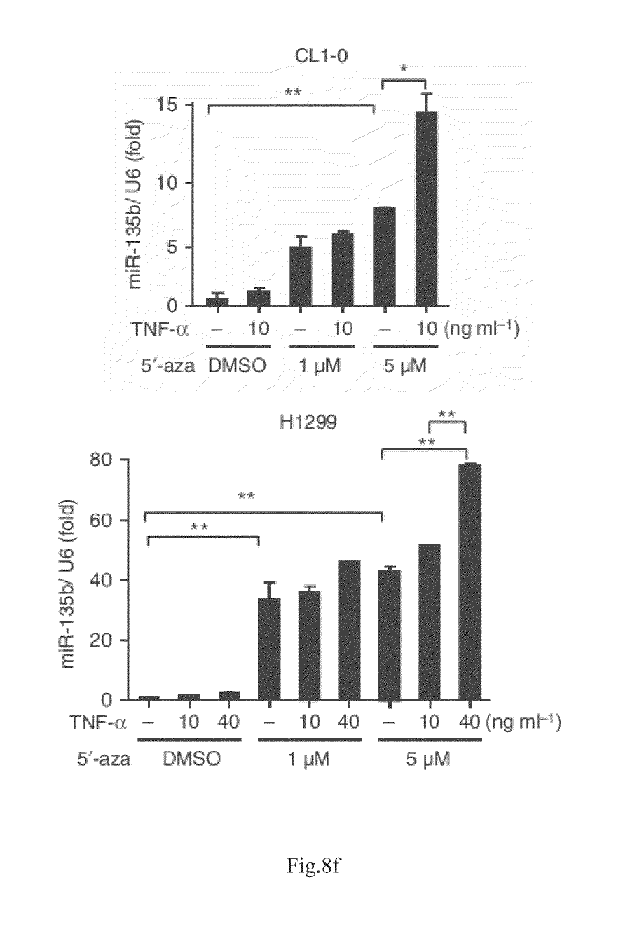

Based on the above-mentioned, miR-135b was identified as an invasion/metastasis modulator. In order to elucidate the mechanism underlying the deregulation of miR-135b, the promoter region of miR-135b was hypomethylated in CL1-5 cells compared with the same region in normal human bronchial epithelial (NBE) and CL1-0 cells, which was found using differential methylation hybridization (DMH) microarray screening. Corresponding to the results of the DMH array, there was a putative CpG island in the miR-135b promoter region. The DMH array results in CL-series lung cancer cell lines were further verified. As shown in FIG. 8a and FIG. 8b, bisulfite sequencing and quantitative methylation-specific PCR (qMS-PCR) of the miR-135b CpG island region confirmed that the methylation levels of the miR-135b promoter regions were lower in the highly invasive lung cancer cell lines. Moreover, as shown in FIG. 8c, the miR-135b expression levels in the cancer cell were restored by the DNA methylation inhibitor 5-aza-2′CdR.

miR-135b is an intronic miRNA located in the intron 1 region of LEMD1. It was hypothesized that the expressions of both genes were driven by the same promoter. The expression levels of miR-135b and LEMD1 in lung cancer cell lines were similar. Additionally, 5-aza-2′CdR treatment restored LEND1 mRNA expression in a dose-dependent manner. Taken together, these results indicated that the promoter region of miR-135b can be epigenetically regulated by DNA methylation in lung cancer cells.

In addition to the DNA methylation results, a putative NF-κB (nuclear factor kappaB) binding site is in the CpG island in the miR-135b promoter region. Thus, the effect of NF-κB activation on miR-135b expression was examined by chromatin immunoprecipitation (ChIP) assay. As shown in FIG. 8d upper panel, the putative NF-κB binding site within the miR-135b promoter was occupied by p65 when Cl1-0 cells were treated with 5-aza-2′CdR. As shown in FIG. 8d lower panel, TNF-α was used to stimulate NF-κB activation, the association of NF-κB with the miR-135b promoter was confirmed. As shown in FIG. 8e, the effect of NF-κB signaling-induced miR-135b expression was also observed in H1299 and CL1-5-F4 cells after treatment with TNF-α. The TNF-α-simulated miR-135b expression was negated by a NF-κB inhibitor BAY-117082, further supporting the role NF-κB activation in mediating the upregulation of miR-135b.

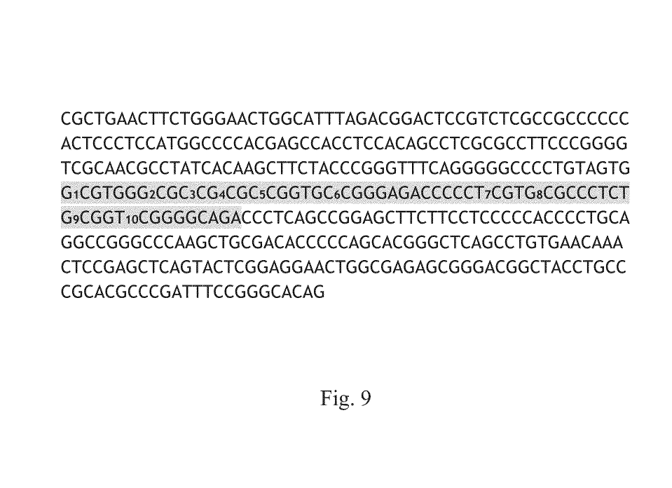

Thus, DNA methylation may prohibit the binding of the NF-κB transactivation complex to the miR-135b promoter region. To evaluate this possibility, the methyaltion levels of the 10 GpC sites (FIG. 9) in the NF-κB binding region of the miR-135b promoter region (SEQ ID NO:15) were measured using a quantitative pyrosequencing assay. The results revealed that the percentage of methylation of the CpG sites of putative NF-κB binding sites in CL1-0 and CL1-1 cells were higher than in CL1-5 and CL1-5-F4 cells, which implied that DNA methylation hindered NF-κB-miR135b binding. To clarify whether DNA methylation and NF-κB worked together to regulate miR-135b transcription, CL1-0 and H1299 cells were co-treated with 5-aza-2′CdR and TNF-α. As shown in FIG. 8f, the expression of miR-135b was dramatically elevated compared with the expression resulting from a single treatment by either molecule alone, whereas LATS2 and LZTS1 were gradually decreased in the H1299 cells, suggesting that miR-135b and its targets are synergistically regulated by these two mechanisms.

In summary, the present invention discloses a novel dual-regulatory mechanism consisting of an epigenetic factor and inflammatory stimulation that synergistically activated oncogenic miR-135b. And the modulation of mi-RNA-135b promoted cancer invasion and metastasis via downregulation of multiple targets in the Hippo pathway and of the tumor suppressor LZTS1. The dysregulation of miR-135b was involved in lung cancer progression in lung cancer progression indicating that a miR-135b antagomir may have a therapeutic potential for cancer treatment.

Those skilled in the art will readily observe that numerous modifications and alterations of the device and method may be made while retaining the teachings of the invention. Accordingly, the above disclosure should be construed as limited only by the metes and bounds of the appended claims.

### REFERENCES

Chu, Y. W., et al. (1997) Selection of invasive and metastatic subpopulations from a human lung adenocarcinoma cell line. Am. J. Respir. Cell Mol. Biol. 17, 353-360
Chan, M. W. Y., et al. (2005) Hypermethylation of 18S and 28S ribosomal DNAs predicts progression-free survival in patients with ovarian cancer. Clin. Cancer Res. 11, 7376-7383

What is claimed is:

1. A method of determining the prognosis of a subject with lung cancer, comprising:
a. measuring the expression level of miRNA-135b in a test sample from a subject with lung cancer, and
b. determining the prognosis of the subject with lung cancer, wherein high expression level of miR-135b in the test sample compared to noncancerous lung tissue control indicates an adverse prognosis.

2. The method according to claim 1, further comprises a step of measuring the expression level of LZTS1, wherein the expression level of LZTS1 in the test sample compared to noncancerous lung tissue control less than 0.25 fold indicates an adverse prognosis.

3. The method according to claim 1, further comprises a step of measuring the expression levels of LZTS1 and LATS2, wherein decreased expression levels of LZTS1 and LATS2 in the test sample compared to noncancerous lung tissue control indicate an adverse prognosis.

4. The method according to claim 1, further comprises a step of measuring at least one additional gene selected from the group consisting of LZTS1, LATS2 and nuclear TAZ expression, wherein decreased expression levels of LZTS1 and LATS2 compared to noncancerous lung tissue control indicate an adverse prognosis, and increased expression level of nuclear TAZ in the test sample compared to noncancerous lung tissue control indicates an adverse prognosis.

5. The method according to claim 1, wherein the lung cancer is non-small-cell lung cancer (NSCLC).

6. The method according to claim 1, wherein a prediction of prognosis is given by a likelihood score derived from using Kaplan-Meier survival analysis, wherein the performance of miRNA-135b of subject is assessed.

7. The method according to claim 2, further comprising a step of performing a Kaplan-Meier survival analysis, wherein the performance of at lest one miRNA-135b and LZTS1 of subject is assessed.

8. The method according to claim 3, further comprising a step of performing a Kaplan-Meier survival analysis, wherein the performance of at least one of miRNA-135b, LZTS1 and LATS2 of subject is assessed.

9. The method according to claim 4, further comprising a step of performing a Kaplan-Meier survival analysis, wherein the performance of at least one of miRNA-135b, LZTS1, LATS2 and nuclear TAZ of subject is assessed.

10. The method according to claim 1, wherein the adverse prognosis indicates growth, invasion, migration and metastasis of lung cancer.

11. A method of inhibiting growth, invasion, migration and metastasis of lung cancer in a subject, which comprises administering the subject with an effective amount of miRNA sponge or miRNA antagomir.

12. The method according to claim 11, wherein the miRNA sponge is miR-135b-specific molecular sponge.

13. The method according to claim 11, wherein the miRNA antagomir is miR-135b-antagomir.

14. The method according to claim 11, wherein the lung cancer is non-small-cell lung cancer (NSCLC).

15. A method of assaying and/or identifying a test agent as a regulator of a methylation level of miRNA-135b for treatment lung cancer, comprising:
a. providing a cell comprising a CpG island of the miRNA-135b promoter region, and treating the cell with the test agent or a vehicle control;
b. measuring the methylation level in the CpG island of the miRNA-135b promoter region, and calculating the ratio of the methylation level of the miRNA-135b promoter region in the presence and the absence of the test agent; and
c. identifying the test agent as a regulator of the methylation level of miRNA-135b when the ratio in the presence of the test agent is more than that in the vehicle control.

16. The method according to claim 15, wherein the lung cancer is non-small-cell lung cancer (NSCLC).

17. The method according to claim 15, wherein the CpG island of miR-135b promoter region contains NF-κB (nuclear factor kappaB) binding site.

18. The method according to claim 15, wherein the step b comprises differential methylation hybridization (DMH) microarray screening, methylation-specific polymerase chain reaction (MSP), quantitative methylation-specific polymerase chain reaction (QMSP), bisulfite sequencing (BS), microarrays, mass spectrometry, denaturing high-performance liquid chromatography (DHPLC), and pyrosequencing.

19. The method according to claim 15, wherein lower level of methylation of CpG island of miRNA-135b promoter region indicates an adverse prognosis.

20. The method according to claim 19, wherein the adverse prognosis indicated growth, invasion, migration and metastasis of lung cancer.
